(19) United States
(12) Patent Application Publication
(10) Pub. No.: US 2010/0068297 A1
(43) Pub. Date: Mar. 18, 2010

(54) Antimicrobial Composition

(75) Inventor: Declan Naughton, Surrey (GB)

Correspondence Address: YOUNG BASILE, 3001 WEST BIG BEAVER ROAD, SUITE 624, TROY, MI, 48084, US

(73) Assignee: NATURE THERAPEUTICS LIMITED, Lincolnshire (GB)

(21) Appl. No.: 12/517,685

(22) PCT Filed: Dec. 6, 2007
(86) PCT No.: PCT/GB2007/050743

§ 371 Date: Jun. 4, 2009

(30) Foreign Application Priority Data
Dec. 6, 2006 (GB) 0624340.6

(51) Int. Cl. A61K 33/34; A61K 33/24; A61P 31/04

(52) U.S. Cl. 424/630; 424/646

(57) ABSTRACT

Disclosed herein are antimicrobial compositions comprising an effective concentration of a metal salt combined with a plant extract. In some embodiments the composition comprises a copper salt and/or an iron salt and/or a nickel salt and/or a cobalt salt; and an extract of a plant selected from a group consisting of Punka granatum, Viburnum plicatum, Camellia sinensis, and Acer spp. The invention extends to uses of such compositions as medicaments, and to methods of treating microbial infections. The invention extends to methods for preventing microbial infections by coating objects and surfaces with the compositions.

### CROSS-REFERENCE TO RELATED APPLICATIONS

This application claims priority from International Patent Application PCT/GB2007/050743, filed on Dec. 6, 2007, and Great Britain Patent Application GB 0624340.6, filed on Dec. 6, 2006, the disclosures of which, including their specifications, drawings and claims, are incorporated herein by reference in their entirety.

### FIELD OF THE INVENTION

The present invention relates to antimicrobial compositions, and to uses of such compositions as medicaments, and in methods of treating microbial infections. The invention extends to methods of preventing microbial infections on objects and surfaces coated with the compositions.

### BACKGROUND

The growth in viral infections along with the emergence of antimicrobial drug resistance in human bacterial pathogens is an increasing problem worldwide. As a consequence, effective treatment and control of such micro-organisms is becoming a greater challenge. However, bacterial resistance has appeared for every major class of antibiotic. Since the introduction of antimicrobials, the emergence of resistance has become increasingly evident, particularly in important pathogens such as E. coli, Salmonella spp., Campylobacter spp., Enterococcus spp and Staphylococcus spp.

Over the last decade, research into the antimicrobial properties of traditional plant-based medicines has increased. These studies have screened numerous plants for antimicrobial properties. For example, Punica granatum L. (Punicaceae), which is referred to in English as pomegranate, has been highlighted in many of these studies as having antimicrobial activity against a range of both Gram-positive and Gram-negative bacteria. Recently, several studies have concentrated on the antimicrobial properties of pomegranates. For example, one group of researchers used different extraction methods using pomegranates against a range of six bacteria, including S. aureus, E. coli, K. pneumoniae, P. vulgaris, B. subtilis and S. typhi, and demonstrated good activity against all isolates tested. Another group demonstrated that pomegranate extract was able to inhibit not only the growth of S. aureus, but also the production of enterotoxin.

Many bacteria have advanced protective mechanisms to detoxify heavy metal ions. However, despite this, a wide range of literature exists describing the development of metal compounds as antimicrobial agents. Many low molecular mass metal compounds exhibit bactericidal and/or bacteriostatic activities. In one study, the susceptibilities of Staphylococcus strains to solutions of metal salts (in the range of 50 mmol to 80 mmol) were determined. The frequencies of resistance for Staphylococcus strains varied widely between different metal salts. Accordingly, despite the growing need for new antimicrobial therapies, the mechanism of action of many metal binding antibiotics is not understood.

The enhancement of antimicrobial activities of various plant extracts by the addition of metal salts has been previously investigated. For example, EP 0,744,896B1 discloses antimicrobial compositions, which are based on a combination of ferrous salts and an extract from a plant such as pomegranate rind, Viburnum plicatum leaves or flowers, tea leaves, or maple leaves. The addition of ferrous salts to the plant extract was found to enhance the anti-viral and anti-fungal activities of the composition.

However, a significant problem with these iron salt-based plant extract compositions is that they lack stability, and therefore retain their antimicrobial activity for only very short periods, i.e. up to a maximum of 30 minutes. Accordingly, these compositions are of limited use. Another problem with these iron-based antimicrobial compositions is that they become discolored upon application to a subject. It will be appreciated that antimicrobial compositions that become discolored are far from ideal in the majority of applications, in particular those for topical use on patients, and also in non-therapeutic uses, such as on surfaces prone to microbial infection in hospitals. A further disadvantage with the iron-based compositions disclosed in EP 0,744,896B1 is that they are optimally active at low pH (i.e. circa pH 4.0). Compositions which are optimally active only in acidic conditions are disadvantageous in the majority of applications, particularly when treating patients. Furthermore, such compositions are particularly difficult to formulate.

It is therefore an object of the present invention to obviate or mitigate one or more of the problems of the prior art, whether identified herein or elsewhere, and to provide improved antimicrobial compositions, which may be used in methods for treating microbial infections.

### BRIEF SUMMARY

Disclosed herein are embodiments of antimicrobial compositions. One such antimicrobial composition comprises a copper salt and/or a cobalt salt and/or a nickel salt; and an extract of a plant selected from a group consisting of Punica granatum, Viburnum plicatum, Camellia sinensis, and Acer spp.

Another embodiment of an antimicrobial composition comprises a copper salt and/or an iron salt and/or a nickel salt and/or a cobalt salt; an extract of a plant selected from a group consisting of Punica granatum, Viburnum plicatum, Camellia sinensis, and Acer spp.; and a reducing agent.

Other embodiments will be described herein.

### BRIEF DESCRIPTION OF THE DRAWINGS

For a better understanding of the invention, and to show how embodiments of the same may be carried into effect, reference will now be made, by way of example, to the accompanying diagrammatic drawings, in which: —

FIG. 1 is a JOB plot of Fe(III)-PRE ratio against absorbance at 563 nm. The JOB plot shows that the isolated PRE active component binds to ferric ions in the ratio of 1:2 (Fe:PRE);

FIG. 2 is a bar chart demonstrating bactericidal efficacy of the PRE-FE(I) mixture on addition of the reducing agent Vitamin C (CFU refers to Colony Forming Units, all CFU/ml values are in log₁₀);

FIG. 3 is a bar chart demonstrating the effects of various metal ions with additions of PRE (all CFU/ml values are in log₁₀);

FIG. 4 is a bar chart showing bactericidal activities for mixtures at 24 and 48 hour: in equates to bactericidal mixture added directly, out refers to mixture prepared and stored for 24 or 48 hours prior to addition (all CFU/ml values are in log₁₀);

FIG. 5 is a bar chart showing a bactericidal assay of pomegranate ointment 1 week after formulation (all CFU/ml values are in log₁₀);

FIG. 6 is a bar chart showing a bactericidal assay of pomegranate ointment 3 weeks after formulation (all CFU/ml values are in log₁₀);

FIG. 7 is a bar chart showing preliminary toxicity screening using Trypan blue staining;

FIG. 8 is a bar chart showing infectious agent survival after 30 minutes exposure to fresh ointment preparations of test agents shown;

FIG. 9 is a bar chart showing infectious agent survival after 30 minutes exposure to ointment preparations of test agents shown after storage at 5° C. for 3 months;

FIG. 10 is a bar chart showing the antimicrobial activities of PRE alone and in combination with metal ions after a 30 minute incubation against Ps. aeruginosa, P. mirabilis and E. coli, using Lambda buffer as a control;

FIG. 11 is a bar chart showing the antimicrobial activities of PRE alone and in combination with metal ions after a 30 minute incubation against S. aureus and B. subtilis, using Lambda buffer as a control;

FIG. 12 is a bar chart showing the antimicrobial activities of PRE/metal ion combinations with the addition of Vitamin C after a 30 minute incubation against all isolates tested, using Lambda buffer as a control (all CFU/ml values are in log₁₀);

FIG. 13 shows Box Whisker statistical analysis of the viable count data achieved in relation to the antimicrobial activities of PRE alone and in combination with Cu(II) ions after a 2 hour minute incubation against 10 clinical isolates of MRSA using Lambda buffer as a control. (Box represents 25% and 75% quartiles, bar represents median and error bars represent range. Mean cfu ml⁻¹ value shown by ▴) (all CFU/ml values are in log₁₀);

FIG. 14 shows Box Whisker statistical analysis of the viable count data achieved in relation to the antimicrobial activities of PRE alone and in combination with Cu(II) ions after a 2 hour minute incubation against 10 clinical isolates of MSSA using Lambda buffer as a control. (Box represents 25% and 75% quartiles, bar represents median and error bars represent range. Mean cfu ml⁻¹ value shown by ) (all CFU/ml values are in log₁₀);

FIG. 15 shows Box Whisker statistical analysis of the viable count data achieved in relation to the antimicrobial activities of PRE alone and in combination with Cu(II) ions after a 2 hour minute incubation against 10 clinical isolates of PVL positive cMRSA using Lambda buffer as a control. (Box represents 25% and 75% quartiles, bar represents median and error bars represent range. Mean cfu ml⁻¹ value shown by ▪) (all CFU/ml values are in log₁₀);

FIG. 16 shows the antimicrobial activities of PRE alone and in combination with Fe(II) or Cu(II) ions and Vitamin C after a 30 minute incubation against 9 clinical isolates of ESL Pseudomonas aeruginosa using Lambda buffer as a control. (Box represents 25% and 75% quartiles, bar represents median and error bars represent range. Mean cfu ml⁻¹ value shown by *) (all CFU/ml values are in log₁₀);

FIG. 17 shows the antimicrobial activities of the ointment formulation of PRE in combination with Fe(II) or Cu(II) ions and Vitamin C after a 30 minute incubation against 9 clinical isolates of ESβL Pseudomonas aeruginosa using Lambda buffer as a control. (Box represents 25% and 75% quartiles, bar represents median and error bars represent range. Mean cfu ml⁻¹ value shown by *) (all CFU/ml values are in log₁₀);

FIG. 18 shows activities of black and green tea extracts alone or in combination with metal salts additives against Staph. aureus using Lambda buffer as a control. Legend: Black tea with iron (BTI), Black tea with copper (BTC), Green tea with iron (GTI), Green tea with copper (GTC). Error bars are SEM for each sample tested (all CFU/ml values are in log₁₀);

FIG. 19 shows activities of black and green tea extracts alone or in combination with metal salts additives against Prot. mirabilis using Lambda buffer as a control (abbreviations as in FIG. 18). Error bars are SEM for each sample tested (all CFU/ml values are in log₁₀); and

FIG. 20 shows activities of black and green tea extracts alone or in combination with metal salts additives against Ps. aeruginosa using Lambda buffer as a control (abbreviations as in FIG. 18). Error bars are SEM for each sample tested. All CFU/ml values are in log₁₀.

### DETAILED DESCRIPTION OF EMBODIMENTS OF THE INVENTION

The inventor based his research on the anti-viral and anti-fungal compositions reported in EP 0,744,896B1 in an attempt to solve the problems inherent with these compositions. In order to address these problems, the inventor investigated whether or not it was possible to substitute the ferrous ions with other metal ions to form an antimicrobial composition, which exhibited improved properties, i.e. did not turn black, had antimicrobial activity for more than 30 minutes, and was active at a more amenable pH, such as neutral pH. Therefore, a number of other metal ions were tested in combination with an active plant extract, for example, pomegranate rind extract (PRE) as a model for other plant extract-based compositions.

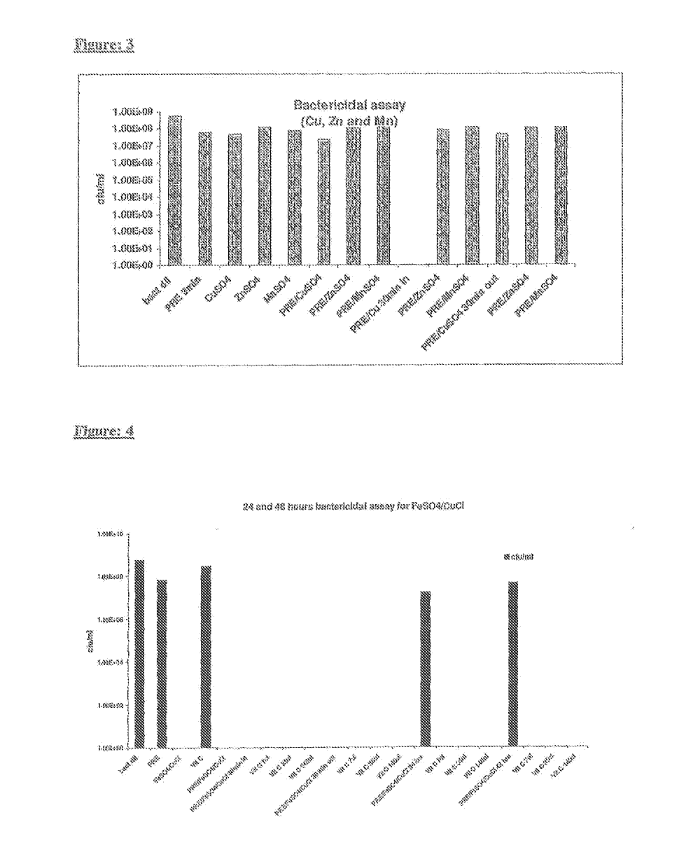

As described in Example 2, the metal ions that were tested for their abilities to enhance the activity of the PRE included copper (I), copper (II), zinc (II), and manganese (II). Iron (II) salts were also tested as a control. As shown in the results in FIG. 2, as expected, the iron (II) compositions exhibited antimicrobial activity, thereby confirming the work disclosed in EP 0,744,896B1. However, the inventor noticed that zinc and manganese ion-based compositions exhibited no antimicrobial activity at all, as shown in FIG. 3. Furthermore, surprisingly, the highest activity was exhibited by Cu(II) salts upon addition to PRE. Given that zinc and manganese-based plant extract combinations are ineffective, but copper-based compositions are active, the inventor has suggested that there is some, as yet unknown, mechanism of action for these metal ion-based antimicrobial compounds.

From a consideration of the Periodic Table, the inventor has noticed a pattern emerge concerning which metal ions are active and which are inactive when combined with plant extracts. Given that manganese- and zinc-based compositions are inactive, and given that iron- and copper-based compositions are active, the inventor believes that salts of the two metals that are between iron and copper in period 4 of the transition elements, i.e. cobalt and nickel, may also be used to prepare active antimicrobial compositions.

The inventor believes that he is the first to prepare antimicrobial compositions based on a combination of a copper salt, or a nickel salt, or a cobalt salt, combined with a suitable plant extract, for example, pomegranate plant extract or tea leaves.

Therefore, according to a first aspect of the invention, there is provided an antimicrobial composition comprising (i) a copper salt and/or a cobalt salt and/or a nickel salt; and (ii) an extract of a plant selected from a group consisting of Punica granatum, Viburnum plicatum, Camellia sinensis, and Acer spp.

The inventor has found that compositions according to the first aspect, which comprise an extract of a plant, such as Punica granatum (ie pomegranate), Viburnum plicatum, Camellia sinensis (ie tea), and Acer spp. combined with salts of copper, nickel and/or cobalt exhibit surprisingly effective antimicrobial activity, and in some cases are more active that known iron-based compositions. This activity could not have been predicted as the mechanism of action is unknown, and not predictable in view of the fact that manganese- and zinc-based compositions are inactive. From his studies, and as demonstrated in the examples, the inventor has found that copper salts appear to be the most active.

Therefore, the composition according to the first aspect can comprise an effective concentration of a copper salt and an effective concentration of an extract of a plant selected from a group consisting of Punica granatum, Viburnum plicatum, Camellia sinensis, and Acer spp.

A significant advantage of such copper-based compositions is that they are less likely to become discoloured, in use, than known iron-based compositions. While the inventor does not wish to be bound by any hypothesis, he believes that known iron-based antimicrobial compositions become discolored because aromatic compounds contained within the composition are polymerized in the presence of the iron ions. However, surprisingly, and advantageously, such polymerization does not occur in the presence of copper ions, and so the composition according to the first aspect does not become discolored. This is a significant advantage of using copper-based compositions over the known iron-based compositions because they may be used in many more applications, which are currently not possible with iron-based compositions, such as topically on patients.

By the term “antimicrobial composition”, we mean a substance or agent, which kills, inhibits or slows the growth of a micro-organism. Examples of micro-organisms, which the composition according to the invention may combat, include bacteria, viruses, fungi, or protozoa, and other pathogens and parasites.

To stabilize and prolong the activity of the antimicrobial compositions according to the first aspect of the invention, and also known iron-based antimicrobial compositions, as described in Example 1, the inventor carried out spectroscopic metal ion binding studies to investigate the mechanism of action of the iron-based composition disclosed in EP 0,744,896B1. Interestingly, the results of the metal binding study indicate that the activation step for enhanced antibiotic activity (i.e. addition of ferrous ions to the PRE component) results in the oxidation of the metal ion from the Fe(II) to the Fe(III) oxidation state. Although the inventor does not wish to be bound by any hypothesis, he believes that the significant loss of activity of the iron-based antimicrobial composition, which is witnessed after 30 minutes, may be directly attributable to this oxidation process.

This surprising realization led the inventor to investigate the effects of adding a reducing agent to the active mixture in an attempt to re-generate the Fe(II) by reduction of the oxidised Fe(III) ions to rejuvenate efficacy, and activity. To test his hypothesis, the inventor chose Vitamin C as a reducing agent (reductant) to see if it had the effect of extending the activity life of iron-based and also copper-based compositions.

As described in the Examples, to his surprise, adding Vitamin C did have a significant effect of considerably extending the activity of both iron- and copper-based plant extract ointment compositions. The results clearly demonstrate that enhanced bactericidal activity (against P. aeruginosa) occurs when Vitamin C was included in both copper- and iron-based compositions.

Therefore, the composition according to the first aspect can comprise a reducing agent. The inventor believes that this stabilizing or activating effect of the reducing agent on the compositions according to the invention is an important aspect of the invention as it not only applies to copper-based plant extract compositions, but also to known iron-based compositions. The inventor also believes that the benefit of adding a reducing agent could be used in relation to cobalt- and nickel-based compositions.

Therefore, according to a second aspect of the invention, there is provided an antimicrobial composition comprising (i) a copper salt and/or an iron salt and/or a nickel salt and/or a cobalt salt; (ii) an extract of a plant selected from a group consisting of Punica granatum, Viburnum plicatum, Camellia sinensis, and Acer spp.; and (iii) a reducing agent.

While the inventor does not wish to be bound by any hypothesis, he believes that the reducing agent has the effect of maintaining the iron in the Fe(II) active state and the copper in the Cu (II) active state. Examples 3, 4 and 7 demonstrate how effective the addition of a reducing agent is to the activity of these compositions. The inventor was surprised that the duration of the activity of iron-based and copper-based compositions, particularly when formulated as ointments, could be increased from 30 minutes in the absence of a reducing agent to as much as 3 months in the presence of a reducing agent. This was totally unexpected, and most advantageous in medical applications.

By the term “reducing agent”, we mean any agent or compound that donates electrons to the metal ion, i.e. copper or iron or cobalt or nickel, in the composition.

The skilled technician will appreciate the various types of reducing agent or reductant that may be combined in the composition. For example, the reducing agent may be cysteine or glutathione. A reducing agent can comprise Vitamin C (i.e. ascorbate), which is shown to be surprisingly active in the Examples.

One, five and twenty equivalents of Vitamin C have been used relative to the metal ion which in the Examples was normally fixed at about 4.8 millimoles. Where a reducing agent is used, the composition can comprise between about 1 mM and about 200 mM reducing agent, between about 2 mM and about 150 mM reducing agent, even between about 3 mM and about 120 mM reducing agent, and between about 4 mM and about 100 mM reducing agent.

FIG. 2 demonstrates that the effect of the reducing agent increases with increasing concentration of Vitamin C. Hence, excess concentrations of reducing agent can be used compared to the metal ion.

Therefore, suitable effective concentrations of the reducing agent in compositions according to the first and second aspect of the invention are in a weight ratio of the reducing agent to the metal ion of at least 1:1, more suitably at least 2:1, and even more suitably at least 5:1. Weight ratios of reducing agent to metal ion can be at least 10:1, at least 20:1, and at least 50:1.

The compositions of the first and second aspect exhibit surprisingly high antimicrobial activities.

In a third aspect, there is provided a composition comprising (i) a copper salt and/or a cobalt salt and/or a nickel salt; and (ii) an extract of a plant selected from a group consisting of Punica granatum, Viburnum plicatum, Camellia sinensis, and Acer spp.; or a composition comprising (i) a copper salt and/or an iron salt and/or a cobalt salt and/or a nickel salt; (ii) an extract of a plant selected from a group consisting of Punica granatum, Viburnum plicatum, Camellia sinensis, and Acer spp.; and (iii) a reducing agent; for use as a medicament.

In a fourth aspect there is provided a composition comprising (i) a copper salt and/or a cobalt salt and/or a nickel salt; and (ii) an extract of a plant selected from a group consisting of Punica granatum, Viburnum plicatum, Camellia sinensis, and Acer spp.; or a composition comprising (i) a copper salt and/or an iron salt and/or a cobalt salt and/or a nickel salt; (ii) an extract of a plant selected from a group consisting of Punica granatum, Viburnum plicatum, Camellia sinensis, and Acer spp.; and (iii) a reducing agent; for use in treating, ameliorating or preventing a microbial infection.

Furthermore, in a fifth aspect, there is provided use of a composition comprising (i) a copper salt and/or a cobalt salt and/or a nickel salt; and (ii) an extract of a plant selected from a group consisting of Punica granatum, Viburnum plicatum, Camellia sinensis, and Acer spp.; or a composition comprising (i) a copper salt and/or an iron salt and/or a cobalt salt and/or a nickel salt; (ii) an extract of a plant selected from a group consisting of Punica granatum, Viburnum plicatum, Camellia sinensis, and Acer spp.; and (iii) a reducing agent; in the manufacture of a medicament for the treatment, amelioration or prevention of a microbial infection.

According to a sixth aspect, there is provided a method of treating, preventing or ameliorating a microbial infection, the method comprising administering to a subject in need of such treatment a therapeutically effective amount of a composition comprising (i) a copper salt and/or a cobalt salt and/or a nickel salt; and (ii) an extract of a plant selected from a group consisting of Punica granatum, Viburnum plicatum, Camellia sinensis, and Acer spp.; or of a composition comprising (i) a copper salt and/or an iron salt and/or a cobalt salt and/or a nickel salt; (ii) an extract of a plant selected from a group consisting of Punica granatum, Viburnum plicatum, Camellia sinensis, and Acer spp., and (iii) a reducing agent.

According to a seventh aspect, there is provided use of a composition comprising (i) a copper salt and/or a cobalt salt and/or a nickel salt; and (ii) an extract of a plant selected from a group consisting of Punica granatum, Viburnum plicatum, Camellia sinensis, and Acer spp.; or a composition comprising (i) a copper salt and/or an iron salt and/or a cobalt salt and/or a nickel salt; (ii) an extract of a plant selected from a group consisting of Punica granatum, Viburnum plicatum, Camellia sinensis, and Acer spp., and (iii) a reducing agent; as an antimicrobial agent.

The inventor has found that the compositions according to the first and second aspects comprising an effective concentration of copper salt and/or iron salt and/or a cobalt salt and/or a nickel salt exhibit antimicrobial activity. An effective concentration of the copper, iron, nickel, or cobalt salt can be in the range of about 0.1 mM to about 200 mM, between about 0.3 mM and about 100 mM, between about 0.5 mM and about 50 mM, between about 1 mM and about 30 mM, and between about 2 mM and about 10 mM.

The nature of the metal salt (i.e. copper salt, iron salt, cobalt salt or nickel salt) is not believed to be critical to the antimicrobial activities of the compositions according to the invention. However, the metal salt can comprise a metal (II) salt.

For example, the nature of the anion in a copper salt is not critical to the efficacy of the antimicrobial composition. However, the results indicate that copper (II) sulfate may be more active than copper (I) chloride. Therefore, where the composition comprises a copper salt, the copper salt can be a copper (II) salt, i.e. a cupric ion or copper sulfate. An effective concentration of the copper salt is in the range of about 0.1 mM to about 200 mM, between about 0.3 mM to about 100 mM, between about 0.5 mM to about 50 mM, between about 1 mM to about 30 mM, and between about 2 mM to about 10 mM.

The plant extract in the composition according to the first aspect can comprise an extract of Punica granatum, and/or pomegranate rind extract (PRE). Therefore, the composition according to the first aspect can comprise copper sulfate, combined with an extract of Punica granatum, and/or PRE. This composition is described herein as copper sulfate/PRE, and has shown considerable advantage over known iron-based compositions as it does not suffer the problem that it turns black in use. In an embodiment where the composition comprises a reducing agent, such as Vitamin C, it is referred to herein as copper sulfate/PRE/Vitamin C.

Furthermore, where the composition according to the second aspect comprises an iron salt, the nature of the anion in the iron salt is not critical to the efficacy of the antimicrobial composition. However, the results demonstrate that iron (II) sulfate exhibits greater antimicrobial activity than iron (III) chloride. Accordingly, the iron salt can be an iron (II) salt, i.e. a ferrous ion or ferrous sulfate. An effective concentration of the iron salt is in the range of about 0.1 mM and about 200 mM, between about 0.3 mM and about 100 mM, between about 0.5 mM and about 50 mM, between about 1 mM and about 30 mM, and between about 2 mM and about 10 mM.

The nature of the anion in a nickel or cobalt salt is also not critical to the efficacy of the antimicrobial composition. However, where the composition comprises a nickel salt or a cobalt salt, the nickel salt can be a nickel (II) salt, and the cobalt salt can be a cobalt (II) salt. The nickel salt can be nickel sulfate, and the cobalt salt can be cobalt sulfate. Effective concentrations of the nickel or cobalt salt are in the range of about 0.1 mM and about 200 mM, between about 0.3 mM and about 100 mM, between about 0.5 mM and about 50 mM, between about 1 mM and about 30 mM, and between about 2 mM and about 10 mM.

In one embodiment, the composition according to the first aspect comprises (i) either a copper salt or a cobalt salt or a nickel salt; and (ii) an extract of a plant selected from a group consisting of Punica granatum, Viburnum plicatum, Camellia sinensis, and Acer spp. However, in an embodiment, the composition of the first aspect can comprise at least two metal ions, or at least all three metal ions in combination with the plant extract.

In one embodiment, the composition according to the second aspect comprises (i) either a copper salt or an iron salt or a cobalt salt or a nickel salt; (ii) an extract of a plant selected from a group consisting of Punica granatum, Viburnum plicatum, Camellia sinensis, and Acer spp.; and (iii) a reducing agent.

However, the composition according to the second aspect comprises at least two metal ions, or at least three, or all four of the metal ions, in combination with an extract of a plant selected from a group consisting of Punica granatum, Viburnum plicatum, Camellia sinensis, and Acer spp. and a reducing agent. Copper and iron salts can be combined with the plant extract.

Accordingly, compositions according to the first or second aspect comprise a copper (II) salt and an iron (II) salt combined with an extract of a plant selected from a group consisting of Punica granatum, Viburnum plicatum, Camellia sinensis, and Acer spp. Other compositions according to the second aspect comprise copper sulphate and iron sulphate combined with an extract of a plant selected from a group consisting of Punica granatum, Viburnum plicatum, Camellia sinensis, and Acer spp. and a reducing agent. The composition can be formulated as an ointment, which is described hereinafter.

The plant extract in the composition according to the second aspect comprises an extract of Punica granatum, and omegranate rind extract (PRE). The reducing agent in the composition according to the second aspect is Vitamin C. Accordingly, compositions according to the second aspect comprise copper sulphate, iron sulphate, and an extract of Punica granatum, and/or PRE, and Vitamin C. This composition is described herein as copper sulphate/iron sulphate/PRE/Vitamin C. This composition exhibits surprising antimicrobial activity, and extended shelf-life when formulated as an ointment.

Compositions according to the invention comprise an extract of a plant selected from Punica granatum, Viburnum plicatum, Camellia sinensis, or Acer spp.

Punica granatum is referred to as pomegranate. The inventor has found that extracts from the whole pomegranate may be effectively used to provide antimicrobial compositions according to the invention. However, compositions according to the invention can comprise an extract from the rind of Punica granatum, i.e. pomegranate rind extract (PRE).

Viburnum plicatum has been shown to have an active component. Compositions according to the invention can comprise an extract from leaves or flowers of Viburnum plicatum.

Camellia sinensis is the taxonomic name given to common tea. Any parts of the tea plant may be used to prepare compositions according to the invention, although the tea leaves are most effective. Teas may be green tea or black tea. As demonstrated in Example 8, the inventors have found that a combination of green tea and black tea has effective antimicrobial properties against Staphylococcus aureus, Pseudomonas aeruginosa and Proteus mirabilis.

Acer spp. refers to a broad genus of maple plant. Compositions according to the invention can comprise an extract from leaves or flowers of Acer spp. Acer species can include Acer pseudoplatanus (UK acer) or Canadian maple plant.

In order to prepare suitable plant extracts for preparing compositions according to the invention, the chosen plant is first comminuted, for example in a solvent, which can then be boiled. An example of a solvent is water. The extract may be fractionated, for example by centrifugation. The fractionated extracts contain an active compound.

Example 8 established that the extraction method for green and black tea can be by boiling at about 100° C. for at least 2 min, at least 4 min, for at least 6 min, and at least 10 min.

The plant extracts may be sterilized, for example by autoclaving, and then allowed to cool and stored at −20° C. A further purification of the plant extract (e.g. pomegranate extract) to a molecular weight cut-off of below about 10,000 Da may be carried out, for example, by membrane ultrafiltration before storage.

The plant extract may be used in a concentrated form. Alternatively, the extract may be diluted as appropriate to its intended use. Typically, about 10 g of dried plant extract may be used in about 150 ml of water. This may give an effective concentration of between about 1 and 99% (w/w) plant extract, between about 2 and 80% (w/w) plant extract, and between about 5 and 50% (w/w) plant extract. Effective compositions according to the invention comprise 1-99% (v/v) of the metal salt solution combined with 99-1% (v/v) of the plant extract. The compositions according to the invention may be in the form of a solid or liquid concentrate.

Hence, in an eighth aspect, there is provided a composition according to either the first or second aspect in the form of a solid or liquid concentrate, for dilution with water.

Due to their increased biological activity, compositions comprising copper and/or iron and/or nickel and/or cobalt salts, a plant extract and, in the case of compositions according to the second aspect a reducing agent, are of utility as antimicrobial agents. Hence, the compositions according to the first and second aspects of the invention may be used in the treatment against any microbial infection, such as a bacterial, viral or fungal infection.

The compositions according to the invention can be antibacterial compositions. The bacterium may be a Gram-positive or a Gram-negative bacterium. For example, bacteria against which the compositions in accordance with the invention are effective may include Firmicutes, which may be Bacilli or Clostridia, for example Clostridium botulinum. Further examples of bacteria against which the compositions are effective may include Bacillales, such as Bacillus subtilis, as demonstrated in Example 5.

The compositions may be effective against Staphylococcus, for example, Staphylococcus aureus. As demonstrated in Example 6, the compositions according to the invention are particularly effective against MRSA (methicillin-resistant S. aureus), MSSA (multiple antibiotic-resistant methicillin-resistant S. aureus) and Panton-Valentine Leukocidin (PVL) producing cMRSA isolates (ie community acquired MRSA, which produce Panton-Valentine leukocidin).

Additional Bacillales with which the compositions are effective include Streptococci, for example, Streptococcus pyogenes or Streptococcus pneumoniae.

Further examples of bacteria against which the compositions in accordance with the invention are effective may include Pseudomonadales, such as Pseudomonas aeruginosa (as demonstrated in the Examples). As demonstrated in Example 7, the compositions according to the invention are effective against multi-drug resistant Pseudomonads (such as extended spectrum β-lactamase Pseudomonas aeruginosa).

Further examples of bacteria against which the compositions are effective may include Gammaproteobacteria, which may be selected from a group consisting of Enterobacteriales, Proteus, Serratai, Pasteurellales, and Vibrionales. As demonstrated in Example 5, suitable Enterobacteriales against which the compositions are effective include Escherichia ssp., such as E. coli. Examples of Proteus against which the compositions are effective include Proteus mirabilis as described in the Examples. Examples of Serratai include Serratia marcescens. Examples of Pasteurellales include Haemophilus influenzae. Examples of Vibrionales include Vibrio cholerae.

Further examples of bacteria against which the compositions according to the invention are effective may include Betaproteobacteria, including Neisseriales, for example, Neisseria gonorrhoeae. Further examples of bacteria against which the compositions are effective may include Delta/epsilon subdivided Proteobacteria, including Campylobacterales, for example Helicobacter pylori. Further examples of bacteria against which the compositions are effective may include Actinobacteria, for example Mycobacterium tuberculosis and Nocardia asteroides.

The compositions and medicament according to the invention may be used for the treatment of a variety of bacterial infections, including: microbial keratitis; conjunctivitis; bronchopulmonary infections, for example, pneumonia; urinary tract infections, for example, cystitis, pyelonephritis; ear, nose, and throat infections, for example, otitis media, sinusitis, laryngitis, diphtheria; skin infections including cellulitis, impetigo, wound infections, botulism, gonorrhoea; septicaemia; peptic and duodenal ulcer; gastritis; Campylobacter infections; Proteus mirabilis infections; meningitis; osteomyelitis; and Salmonellosis.

The compositions according to the invention may be antiviral compositions. Compositions and medicaments according to the invention may be used in the treatment of a number of viral infections. The virus may be any virus, and particularly an enveloped virus. Examples of viruses against which the compositions are effective include poxviruses, iridoviruses, togaviruses, or toroviruses. Further examples include a filovirus, arenavirus, bunyavirus, or a rhabdovirus. Further examples include a paramyxovirus or an orthomyxovirus. It is envisaged that virus may be a hepadnavirus, coronavirus, flavivirus, or a retrovirus. The virus may be a herpes virus or a lentivirus. The virus may be Human Immunodeficiency Virus (HIV), Human herpes simplex virus type 2 (HSV2), or Human herpes simplex virus type 1 (HSV1). Alternatively, viruses which may be combated also include bacteriophages.

Compositions according to the invention may be antifungal compositions. Compositions and medicaments according to the invention may be used in the treatment of a number of fungal infections. For example, fungi against which the compositions in accordance with the invention are effective may include a filamentous fungus, such as an Ascomycete. Examples of fungi against which the compositions in accordance with the invention are effective may be selected from a group of genera consisting of Aspergillus; Blumeria; Candida; Cryptococcus; Encephalitozoon; Fusarium; Leptosphaeria; Magnaporthe; Phytophthora; Plasmopara; Pneumocystis; Pyricularia; Pythium; Puccinia; Rhizoctonia; richophyton; and Ustilago.

Further examples of fungi may be selected from a group of genera consisting of Aspergillus and Candida. The fungus may be selected from a group of species consisting of Aspergillus flavus; Aspergillus fumigatus; Aspergillus nidulans; Aspergillus niger; Aspergillus parasiticus; Aspergillus terreus; Blumeria graminis; Candida albicans; Candida cruzei; Candida glabrata; Candida parapsilosis; Candida tropicalis; Cryptococcus neoformans; Encephalitozoon cuniculi; Fusarium solani; Leptosphaerianodorum; Magnaporthe grisea; Phytophthora capsici; Phytophthora infestans; Plasmopara viticola; Pneumocystis jiroveci; Puccinia coronata; Puccinia graminis; Pyricularia oryzae; Pythium ultimum; Rhizoctonia solani; Trichophytoninterdigitale; Trichophyton rubrum; and Ustilago maydis. Further examples of fungi include yeast, such as Saccharomyces spp, eg S. cerevisiae, or Candida spp, and C. albicans, which is know to infect humans.

It will be appreciated that the compositions according to the invention may be used in a monotherapy (ie use of the compositions according to the invention alone to prevent and/or treat a microbial infection or contamination). Alternatively, the compositions according to the invention may be used as an adjunct to, or in combination with, known antimicrobial therapies. For example, conventional antibiotics for combating bacterial infections include amikacin, amoxicillin, aztreonam, cefazolin, cefepime, ceftazidime, ciprofloxacin, gentamicin, imipenem, linezolid, nafcillin, piperacillin, quinopristin-dalfoprisin, ticarcillin, tobramycin, and vancomycin. For example, compounds used in antiviral therapy include acyclovir, gangcylovir, ribavirin, interferon, anti-HIV medicaments including nucleoside, nucleotide or non-nucleoside inhibitors of reverse transcriptase, protease inhibitors and fusion inhibitors. Hence, compositions and medicaments according to the invention may be used in combination with such antibacterial and antiviral agents. Conventional antifungal agents include, for example, farnesol, clotrimazole, ketoconazole, econazole, fluconazole, calcium or zinc undecylenate, undecylenic acid, butenafine hydrochloride, ciclopirox olaimine, miconazole nitrate, nystatin, sulconazole, and terbinafine hydrochloride.

Compositions according to the invention may have a number of different forms depending, in particular, on the manner in which the composition is to be used. Thus, for example, the composition may be in the form of a powder, tablet, capsule, liquid, ointment, gel, hydrogel, aerosol, spray, micellar solution, transdermal patch, liposome suspension or any other suitable form that may be administered to a person or animal. It will be appreciated that the vehicle of the composition of the invention should be one which is well tolerated by the subject to whom it is given.

Compositions and medicaments comprising metal ions, plant extract, and reducing agent (for the second aspect) according to the invention may be used in a number of ways. For instance, oral administration may be required in which case the metal ion, plant extract, and where used, a reducing agent, may be contained within a composition that may, for example, be ingested orally in the form of a tablet, capsule or liquid. Alternatively, the composition may be administered systemically by injection into the blood stream. Injections may be intravenous (bolus or infusion) or subcutaneous (bolus or infusion). The compositions may also be administered by inhalation (e.g. intranasally). Alternatively, compositions according to the invention may be administered by aerosol, for example using an atomizer, by which the composition may be administered nasally or via the lungs.

However, the compositions may be topically applied, for example in the form of an ointment, cream or gel or aqueous solution. Topical administration is useful when a subject to be treated has a microbial skin infection. For instance, ointments may be applied to the skin, areas in and around the mouth or genitals to treat specific viral infections. The composition may be applied intravaginally (for example, if required to protect the subject from sexually transmitted diseases), or rectally. Intravaginal administration is effective for treating sexually transmitted diseases (including AIDS). Topical application to the skin is particularly useful for treating viral infections of the skin or as a means of transdermal delivery to other tissues also.

Example 3 describes the inventor's efforts to produce a formulation for the compositions and medicaments according to the invention. The inventor set out to enhance the stability of the product formulation and conducted preliminary toxicity tests. In order to optimize activity and enhance stability, a number of formulations were prepared and investigated. The antimicrobial activities were screened for a variety of formulations (ie cream, aqueous and ointment formulation). Hence, the composition of the invention may take the form of a cream or aqueous (water-based) solution.

The term “cream” refers to a soft cosmetic-type preparation. Creams of the oil-in-water (O/W) type include preparations such as foundation creams, hand creams, shaving creams, and the like. Creams of the water-in-oil (W/O) type include cold creams, emollient creams, and the like. Pharmaceutically, creams are solid emulsions containing suspensions or solutions of active ingredients for external application.

However, to his surprise, the inventor found that even though the cream and aqueous-based formulations exhibited useful antimicrobial activities, they did not remain as active for as long as the ointment formulation. For reasons not fully understood by the inventor, the activity of the ointment formulation was significantly prolonged compared to that of the cream or aqueous formulation. Hence, the compositions and medicaments in accordance with the invention can be provided as an ointment formulation.

By the term “ointment formulation”, we mean a viscous, semi-solid preparation suitable for topical use on a variety of body surfaces (e.g. the skin). It will be appreciated that an ointment has an oil base containing a substantially high concentration of lipids, and therefore tends to be immiscible in water, whereas a cream has a lower concentration of lipids, and tends to be water soluble. Hence, ointments are more occlusive than creams, and form a protective film over the skin. Ointments are therefore generally composed of single-phase hydrophobic bases, for example of pharmaceutical grades of soft paraffin or microcrystalline paraffin wax. Ointments are generally used for the application of insoluble of oil-soluble medicaments and leave a greasy film on the skin, inhibiting loss of moisture and encouraging hydration of the keratin layer (Physiochemical Principles of Pharmacy by Florence & Attwood, 1992). Ointments should be of such composition that they soften, but not necessarily melt, when applied to the body. They serve as vehicles for the topical application of the active ingredients and may also function as protectives and emollients for the skin.

Accordingly, the ointment formulation may comprise a substantially high concentration of lipid or fat. Suitably, the ointment formulation comprises at least 0.1% (w/w) lipid, more suitably at least 0.5% (w/w) lipid, even more suitably at least 1% (w/w) lipid, and most suitably at least 2% (w/w) lipid. The ointment formulation can comprise at least 5% (w/w) lipid, more suitably at least 10% (w/w) lipid, even more suitably at least 15% (w/w) lipid, and most suitably at least 20% (w/w) lipid. These concentrations are higher than those for cream formulations.

The composition according to the invention may be formulated with an ointment base, which can be hydrous. The ointment base may comprise “wool alcohol ointment” which will be known to the skilled technician. One example of a suitable ointment base which may be used in the preparation of an ointment formulation according to the invention is shown in Table 1.

TABLE 1
Composition of hydrous ointment base (for 60 grams)

| Component | Amount |
|---|---|
| Wool alcohol ointment | 30 g |
| Phenoxyethanol | 0.6 g |
| Dried magnesium sulfate | 0.3 g |
| Purified water, freshly boiled and cooled | 29.1 g |

An ointment formulation may be prepared as follows. The hydrous ointment base may be prepared by mixing the wool alcohol ointment, phenoxyethanol and magnesium sulfate. The purified water shown in the Table was used in the Examples as a control where no plant extract, metal salt or Vitamin C was added. However, in order to prepare an active ointment formulation according to the invention, the plant extract (e.g. PRE) and metal salt is mixed with the ointment base instead of the water, to thereby form the ointment formulation. In embodiments where a reducing agent is used, a solution of a suitable reducing agent (e.g. Vitamin C) may also be added to the ointment base to prepare the active ointment formulation. For example, about 0.072 g of CuSO₄, and about 5.0724 g of Vitamin C may be added to the 29.1 g of PRE which is then added to the ointment base to form the active ointment formulation.

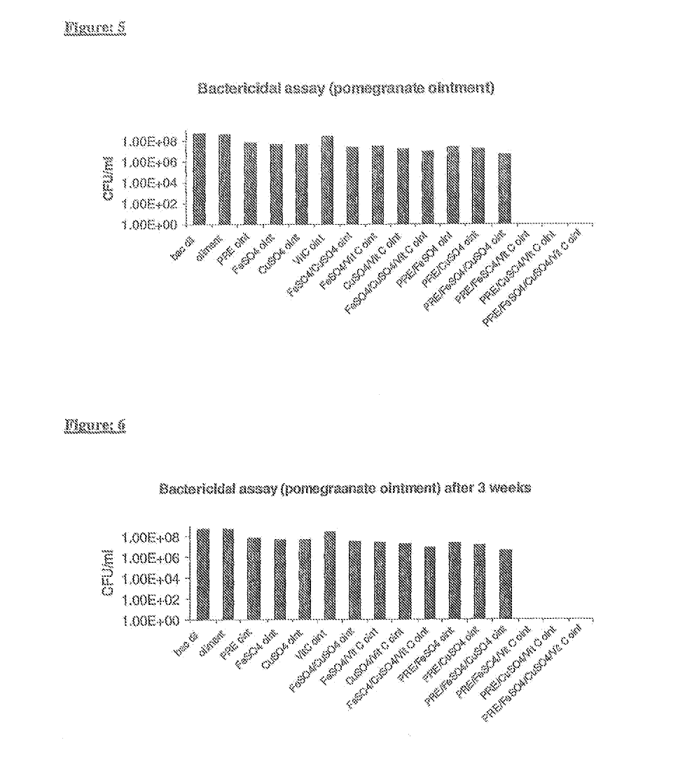

FIG. 5 and Examples 3 and 7 show a bactericidal assay of an ointment of PRE combined with various components, such as iron or copper salts, and Vitamin C. FIG. 6 shows the same compositions, and their activity after 3 weeks. As shown in FIGS. 5 and 6, the activity enhancement upon addition of Vitamin C for either Fe(II)/PRE or Cu(II)/Pre compositions is retained for three weeks with no reduction in efficacy for the ointment formulation. Hence, the ointment formulation shown in Table 1, combined with added Vitamin C exhibited greatly enhanced stability compared to the aqueous preparation (data not shown) showing full retention of activity after 3 weeks. Surprisingly, these findings are in contrast to the cream or aqueous formulations, which in the former case had poor activity and in the latter case lost activity after only 30 minutes. Therefore, the inventor has clearly demonstrated the surprising efficacy, and retained activity over long periods of time, of ointment-based formulations for compositions according to the invention. While the inventor does not wish to be bound by any hypothesis, he believes that the components of the ointment may have some protecting effect on the reducing agent (ie Vitamin C), perhaps preventing it from being oxidized, thereby prolonging its activity on the metal ion and plant extract.

Accordingly, medicaments according to the invention can comprise an ointment formulation comprising copper sulfate/PRE/Vitamin C; or iron sulfate/PRE/Vitamin C; or copper sulfate/iron sulfate/PRE/Vitamin C.

The inventor then carried out toxicity studies on mammalian cell cultures as described in Example 3. These studies showed that the highest percentage of viable cells was observed with PRE while the lowest was encountered after treating the cells with PRE/FeSO₄/CuSO₄/Vitamin C combination which was demonstrated to be non-toxic.

The compositions according to the invention may also be incorporated within a slow or delayed release device. Such devices may, for example, be inserted on or under the skin, and the active compounds (i.e. the iron or copper ion, the plant extract and, where applicable, the reducing agent) may be released over weeks or even months. Such devices may be particularly advantageous when long term treatment with a composition according to the invention is required and which would normally require frequent administration (e.g. at least daily injection).

It will be appreciated that the amount of composition that is required is determined by the biological activity and bioavailability of the active components, which in turn depends on the mode of administration, the physicochemical properties of the composition employed and whether the composition is being used as a monotherapy or in a combined therapy. The frequency of administration will also be influenced by the above-mentioned factors and particularly the half-life of the composition and active agents thereof within the subject being treated.

Optimal dosages to be administered may be determined by those skilled in the art, and will vary with the particular composition in use, the strength of the preparation, the mode of administration, and the advancement of the disease condition, i.e. the microbial infection or contamination. Additional factors depending on the particular subject being treated will result in a need to adjust dosages, including subject age, weight, gender, diet, and time of administration.

Known procedures, such as those conventionally employed by the pharmaceutical industry (e.g. in vivo experimentation, clinical trials, etc.), may be used to establish specific formulations of the compositions according to the invention and precise therapeutic regimes (such as daily doses of the compositions and the frequency of administration).

Generally, a daily dose of between 0.01 μg/kg of body weight and 0.5 g/kg of body weight of compositions according to the invention may be used for the prevention and/or treatment of a microbial infection, depending upon which composition is used. The daily dose can be between 0.01 mg/kg of body weight and 200 mg/kg of body weight, and between approximately 1 mg/kg and 100 mg/kg.

Daily doses may be given as a single administration (e.g. a single daily injection). Alternatively, the composition used may require administration twice or more times during a day. As an example, compositions according to the invention may be administered as two (or more, depending upon the severity of the condition) daily doses of between 25 mg and 7000 mg (i.e. assuming a body weight of 70 kg). A patient receiving treatment may take a first dose upon waking and then a second dose in the evening (if on a two-dose regime) or at 3- or 4-hourly intervals thereafter. Alternatively, a slow release device may be used to provide optimal doses to a patient without the need to administer repeated doses.

Compositions according to the invention generally comprise a pharmaceutically acceptable vehicle.

The invention also provides, in a ninth aspect, a process for making the composition according to the first aspect, the process comprising combining a therapeutically effective amount of a copper salt and/or a cobalt salt and/or a nickel salt, with an extract of a plant selected from a group consisting of Punica granatum, Viburnum plicatum, Camellia sinensis, and Acer spp.; and a pharmaceutically acceptable vehicle.

The invention also provides, in a tenth aspect, a process for making the composition according to the second aspect, the process comprising combining a therapeutically effective amount of a copper salt and/or an iron salt and/or a cobalt salt and/or a nickel salt, with an extract of a plant selected from a group consisting of Punica granatum, Viburnum plicatum, Camellia sinensis, and Acer spp., and a reducing agent; and a pharmaceutically acceptable vehicle.

A “therapeutically effective amount” is any amount which, when administered to a subject, provides prevention and/or treatment of a specific medical condition.

A “subject” may be a vertebrate, mammal, domestic animal or human being.

A “pharmaceutically acceptable vehicle” as referred to herein is any combination of known compounds known to those skilled in the art to be useful in formulating pharmaceutical compositions.

The amount of the composition used may be from about 0.01 mg to about 800 mg. The amount of the composition can be from about 0.01 mg to about 500 mg, about 0.01 mg to about 250 mg, from about 0.1 mg to about 60 mg, and from about 0.1 mg to about 40 mg.

The vehicle may include one or more substances which act as flavoring agents, lubricants, solubilizers, suspending agents, fillers, glidants, compression aids, binders or tablet-disintegrating agents. The vehicle can also be an encapsulating material. In powders, the vehicle may be a finely divided solid that is in admixture with the finely divided active metal salt, plant extract and for the composition of the second aspect, a reducing agent. In tablets, the metal salt, plant extract, and reducing agent may be mixed with a vehicle having the necessary compression properties in suitable proportions and compacted in the shape and size desired. The powders and tablets can contain up to 99% of the active metal ion, plant extract and reducing agent. Suitable solid vehicles include, for example calcium phosphate, magnesium stearate, talc, sugars, lactose, dextrin, starch, gelatin, cellulose, polyvinylpyrrolidine, low melting waxes and ion exchange resins.

Compositions according to the invention may have the form of solutions, suspensions, emulsions, syrups, elixirs and pressurized compositions. The active agents may be dissolved or suspended in a pharmaceutically acceptable liquid vehicle such as water, an organic solvent, a mixture of both, or pharmaceutically acceptable oils or fats. The liquid vehicle may contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid vehicle for oral and parenteral administration include water (containing additives as above, e.g. cellulose derivatives, alcohols (including monohydric alcohols and polyhydric alcohols, e.g. glycols) and their derivatives, and oils (e.g. fractionated coconut oil and arachis oil). For parenteral administration, the vehicle may be an oily ester such as ethyl oleate and isopropyl myristate. Sterile liquid vehicles are useful in sterile liquid-form compositions for parenteral administration. The liquid vehicle for pressurized compositions can be a halogenated hydrocarbon or other pharmaceutically acceptable propellant.

Liquid pharmaceutical compositions which are sterile solutions or suspensions can be utilized by, for example, intramuscular, intrathecal, epidural, intraperitoneal, intravenous and particularly subcutaneous injection. The metal ion combined with plant extract and reducing agent (for the composition of the second aspect) may be prepared as a sterile solid composition that may be dissolved or suspended at the time of administration using sterile water, saline, or other appropriate sterile injectable medium.

Compositions according to the invention can be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. Compositions according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

The compositions according to the invention may be used to treat any mammal, for example, human, livestock, pets, and may be used in other veterinary applications.

The inventor has realized that the compositions according to the invention may be used as a medicament, but may also be put to a number of other antimicrobial uses (whether in a clinical context or otherwise). For instance, in addition to administering the compositions according to the invention to a patient or subject, they may be used for the application to, or coating of, surfaces and objects to prevent, ameliorate or treat microbial infections or contamination.

Therefore, according to an eleventh aspect, there is provided a method of preventing and/or treating a microbial infection or contamination, the method comprising applying to an object or a surface with an amount of a composition that is effective for killing or preventing growth of micro-organisms, wherein the composition comprises (i) a copper salt and/or a nickel salt and/or a cobalt salt; and (ii) an extract of a plant selected from a group consisting of Punica granatum, Viburnum plicatum, Camellia sinensis, and Acer spp.; or the composition comprises (i) a copper salt and/or an iron salt and/or a cobalt salt and/or a nickel salt; (ii) an extract of a plant selected from a group consisting of Punica granatum, Viburnum plicatum, Camellia sinensis, and Acer spp.; and (iii) a reducing agent.

In a twelfth aspect, there is provided an object coated with a composition according to the first or second aspect.

It will be appreciated that the compositions may be particularly useful for application to, or coating of, surfaces or objects that are required to be aseptic. As discussed above, the compositions according to the invention have the advantage that they are antiviral and/or antibacterial and/or antifungal. Accordingly, the compositions disclosed herein have a broad antimicrobial effect. Furthermore, as discussed in more detail below, the compositions may adhere to surfaces and are thereby effective for longer periods of time.

The compositions according to the invention may be used for application to, or coating of, any object or device which is used in a biological or medical situation, such as a medical device, and for which it may be important to prevent a microbial infection or contamination that may lead to any infection in a patient. Examples of medical devices to which compositions according to the invention may be applied include lenses, contact lenses, catheters, stents, wound healing dressings, contraceptives, surgical implants and replacement joints.

The compositions are particularly useful for coating biomaterials and objects and devices made therefrom. Microbial contamination/infection of biomaterials can be particularly problematic because the microbe may use such material as a substrate for growth. Biomaterials (e.g. collagens and other biological polymers) may be used to surface artificial joints. Alternatively, certain implants may substantially comprise such biomaterials.

The compositions may be used to coat surfaces in environments that are required to be aseptic. For instance the compositions may be used in medical environments. The compositions may be used to keep hospital wards clean. They may be used to clean surfaces of medical equipment (e.g. operating tables) in hospitals, such as operating theatres as well as operating theatre walls and floors. The inventor believes the compositions will be useful to improve sterility in general and also to address the spread of MRSA in particular (the inventor believes that MRSA may be killed by the compositions of the invention).

Therefore, the method according to the eleventh aspect may comprise applying the composition to a surface that is selected from: hospital ward surfaces, operating theatre surfaces, kitchen surfaces and sanitary surfaces. It will be appreciated that the above list of objects and surfaces to which compositions according to the invention may be applied is not exhaustive. Hence, the compositions may be administered to any surface, which is prone to a bacterial contamination, for example kitchen and bathroom surfaces and products, such as a toilet seat, or the toilet itself.

The compositions may be formulated into solutions for cleaning objects and surfaces, or for spraying thereon, or in which the object or surface may be immersed. For instance, they may be a routine constituent of physiological solutions (for example as a constituent of physiological saline). Coating of the object or surface may be carried out by preparing an aqueous solution at an appropriate pH and temperature for the composition according to the invention to retain its antimicrobial activity. The object or surface is exposed to the composition for sufficient time to allow immobilization or absorption of a suitable quantity of the composition to the surface thereof or to kill the micro-organism.

Furthermore, the compositions according to the invention may be used to minimize, prevent or treat microbial infections or contamination, by use as, or in conjunction with, a preservative. Hence, the compositions may be used as a preservative in foodstuffs. In addition, the compositions may be used to minimize or prevent microbial growth in cultures, for example in tissue culture work, either to supplement or to replace antibiotics.

All of the features described herein (including any accompanying claims, abstract and drawings), and/or all of the steps of any method or process so disclosed, may be combined with any of the above aspects in any combination, except combinations where at least some of such features and/or steps are mutually exclusive.

### EXAMPLES

The inventor based his initial experiments on the antiviral, anti-bacteriophage, and antifungal compositions disclosed in EP 0,744,896B1. These antimicrobial compositions all include a combination of ferrous salts and an extract from a plant selected from pomegranate rind, Viburnum plicatum leaves or flowers, tea leaves, or maple leaves.

As an example only, the inventor of the present invention focused his research on compositions using pomegranate rind extract (PRE) as the active ingredient. A significant problem with iron salt-based PRE compositions is that they lack stability, and therefore retain their antimicrobial activity for up to a maximum of only 30 minutes. Another problem with these iron-based antimicrobial compositions is that they turn black because aromatics contained within the composition are polymerized in the presence of the iron ions. Accordingly, the focus of the present invention was to develop a stable antimicrobial formulation of the unstable anti-viral and anti-fungal mixture reported in EP 0,744,896B1. Having managed to achieve this goal, the inventor extended his research to investigate and further develop other active antimicrobial compositions. The research is described in the following examples.

### Example 1

The inventor's initial objectives were to set up an in vitro model for screening, isolating and characterizing the active compound(s) in Pomegranate Rind Extract (PRE) in the antimicrobial composition disclosed in EP 0,744,896B1. It was also an aim to investigate the currently unknown mechanism of action of these available compositions in order to assist in the development of new formulations with longer term stabilities.

Materials and methods used for isolating the active compound PRE, and for preparing the iron-based antimicrobial compositions are disclosed in EP 0,744,896B1, which is incorporated herein by reference.

### Preparation of Plant Extracts

Pomegranate rind, Viburnum plicatum leaves or flowers, maple leaves and commercial tea leaves were blended in distilled water (25% w/v), and boiled for about 10 min. After centrifugation (20,000×g, 4° C., 30 min), supernatants were autoclaved (121° C., 15 min), cooled and stored at −20° C. A further purification of the pomegranate extract to a molecular weight cut-off of 10,000 Da was achieved by membrane ultrafiltration and the filtrate stored as above.

### Bacteria, Viruses, and Fungi

The control strain was Pseudomonas aeruginosa for standard experimentation, ie determining optimal preparations. After optimization, the inventor demonstrated activities for the ointment against 10 multidrug-resistant Pseudomonas aeruginosa.

Growth conditions: on nutrient agar provided by Oxoid Ltd for 24 hrs at 37° C.

The inventor established a functional in vitro bactericidal assay to screen a variety of formulations (cream, aqueous and ointment). This assay involved adding 0.5 g of ointment to 10 ml of water and vortexing prior to a standard suspension test using 50 microlitres of bacterial cell suspension to a turbidity of 0.5 McFarland solution (bacterial cell suspension equal to 1.5×10⁸) plus 100 microlitres of the ointment solution. After incubation in the dark at room temperature for 30 mins, serial dilutions (from 10⁻¹ to 10⁻⁵) were carried out on nutrient agar.

Metal binding studies were performed on the isolated component to inform on stability issues for the final formulation and for elucidation of the mechanism of action. A JOB plot was conducted by tracking the maximum wavelength over the mole ration of 0 to 1 for Fe ions and PRE active component.

### Results

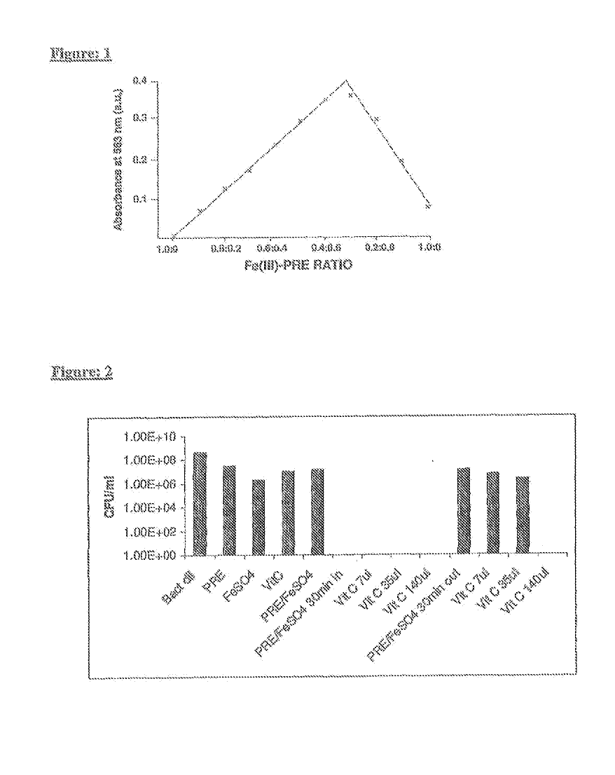

Referring to FIG. 1, there is shown a JOB plot, which captures the results of the spectroscopic metal ion binding studies. The Figure demonstrates: —

Ferric ions (i.e. iron(III) compounds) bind to the active component of PRE giving a characteristic peak at 563 nm attributable to a characteristic Fe(III)-Phenolate complex;

Ferrous ions (i.e. iron(II) compounds) bind to the active component also giving a characteristic peak at 563 nm indicative of oxidation of the metal ion to the Fe(III) state; and

FIG. 1 shows that the isolated PRE active component binds to ferric ions in the ratio of 1:2 (Fe:PRE).

Interestingly, the metal binding study results indicate that the activation step for enhanced antibiotic activity (i.e. addition of ferrous ions to the PRE component) results in the oxidation of the metal ion from the Fe(II) to the Fe(III) oxidation state. Although the inventor does not wish to be bound by any hypothesis, he believes that the significant loss of activity of the iron-based antimicrobial compositions, which is witnessed after 30 minutes, may be directly attributable to this oxidation process.

This surprising realization led the inventor to investigate the effects of adding a reducing agent to the active mixture in an attempt to re-generate the Fe(II) by reduction of the oxidized Fe(III) ions to rejuvenate efficacy, and activity. To this end, the inventor's studies focused on elucidation of the mechanism of action with a view to stabilizing the combined extract (Ferrous salts and PRE). To test his hypothesis, the inventor chose Vitamin C as a reducing agent or reductant to see if it had the effect of extending the activity life of iron-based compositions.

Preliminary results demonstrated enhanced bactericidal activity (on Pseudomonas aeruginosa) occurs on addition of an extra component Vitamin C (a reducing agent added to maintain the iron in the Fe(II) active state). Surprisingly, this tri-component formulation (PRE+Fe(II)+Vitamin C) exhibited exemplary bactericidal activities under the conditions used, as shown in FIG. 2.

Referring to FIG. 2, there is shown the bactericidal efficacy of the PRE-Fe(II) mixture on addition of the reducing agent Vitamin C. As can be seen in the Figure, the value of Colony Forming Units (CFU)/ml is significantly reduced when the PRE/Fe(II) mixture is added immediately upon preparation. However, after 30 minutes, this preparation has lost activity, which is completely restored upon addition of Vitamin C (140 μl).

### Example 2

Based on the surprising findings of Example 1, the inventor then set out to investigate the mechanism of action of the iron-based/PRE compositions at the molecular level with a view to enhancing the product formulation. The enhanced activity upon addition of ferrous ions is problematic as the mixture retains activity for short periods (<30 mins), and principally at low pH values, which is difficult to formulate.

In order to overcome these shortcomings, the inventor investigated whether or not it was possible to substitute the ferrous ions completely with other metal ions, and a number of other metal ions were therefore tested. In addition, based on the positive results seen in Example 1 with addition of a reducing agent (such as Vitamin C), the inventor wanted to see if it was possible to prolong the activity of antimicrobial compositions using other active metal ions by addition of a reducing agent. Finally, the inventor set out to optimize concentrations of the various components in the various active antimicrobial compositions.

### Materials and Methods

It had already been demonstrated that iron-based compositions combined with PRE exhibited antiviral and antifungal activities. Hence, a large range of other metal ions were tested for their abilities to enhance the activity of the PRE, including: —Cu(II), Fe(II), Cu(I), Zn(II) and Mn(II).

### Results

The test solutions of the ions Fe(III), Cu(II), Fe(II), Cu(I), Zn(II) and Mn(II) revealed that the highest activities were exhibited for Fe(II) and Cu(II) species upon addition to PRE as shown in FIGS. 2 and 3. In contrast, as shown in FIG. 3, surprisingly, addition of solutions of Zn(II) and Mn(II) exhibited little or no activity (ie no significant difference from controls).

FIG. 2 demonstrates that the extent of bacterial growth decreased in proportion to the dose of reducing agent, Vitamin C, for each of the following compositions: PRE/FeSO₄. While the inventor does not wish to be bound by any hypothesis, he believes that adding Vitamin C to PRE/FeCl₃ transformed the iron from the ferric state (ie Fe III) to the ferrous state (i.e. Fe II), the latter being more active, and this resulted in a much lower level of bacterial growth compared to the ferric state.

In terms of mechanisms of action, the preference for using metal ions in the reduced state led to the incorporation of studies using reductants to stabilize this oxidation state, and prolong and enhance activity. Thus, the reductant Vitamin C was added in three different doses after pre-incubating the PRE/metal ions mixtures for 30 minutes, and the results are shown in FIG. 2.

FIG. 3 demonstrates that the extent of bacterial growth decreased in proportion for each of the following compositions: CuSO4, ZnSO4, MnSO4, PRE/CuSO₄, PRE/ZnSO₄, PRE/MnSO₄, (‘30 mins in’ refers to addition immediately upon preparation, ‘30 mins out’ refers to a premix and 30 minutes lapse before addition).

The longer term activities of the PRE:metal salt(s) mixtures were investigated after a period of 24 and 48 hours, and the results are shown in FIG. 4. FIG. 4 shows bactericidal activities for mixtures at 24 and 48 hour, in which “in” equates to bactericidal mixture added directly, and “out” refers to mixtures prepared and stored for 24 or 48 hours prior to addition. The results shown are for one system only as an example.

FIG. 3 shows that the activity of the PRE/FeSO₄ system is pronounced at the zero time point. However, even with addition of CuCl to this system, after storage for 48 hrs, considerable bacterial growth was observed as shown in FIG. 4. Surprisingly, full bactericidal activity is restored upon addition of Vitamin C at all concentrations.

### Example 3

The activity of the unstable formulation previously disclosed in EP 0,744,896B1 was tested after storage for several months. The inventor found that this known formulation exhibited no bactericidal activity after only a short period of time. Therefore, following on from the promising results of Example 2, the inventor then set out to enhance the stability of the product formulation and conduct preliminary toxicity tests. In order to optimize activity and enhance stability, a number of formulations were prepared and investigated. The antimicrobial activities were screened for a variety of formulations (i.e. cream, aqueous and ointment). The potential for enhancing efficacy by altering the relative concentrations of active constituents was also explored. Preliminary toxicity tests were conducted on mammalian cell cultures in vitro.

### Materials and Methods

The key objective was to develop a formulation that retained activity to produce an OTC preparation for commercial uses.

For the ointment formulation, a hydrous ointment base was used, the components of which are shown in Table 1. In order to prepare a control ointment formulation having no actives, the magnesium sulfate (0.3 g) and phenoxyethanol (0.6 g) were first dissolved in the purified water (29.1 g) and warmed to 60° C. The wool alcohol ointment was then melted on a separate water bath. The two temperatures were kept the same, and the water solution containing the magnesium sulfate and phenoxyethanol was added in small aliquots to the ointment solution, stiffing constantly until a smooth mix was formed, whilst maintaining the temperature at 60° C. When all the water was added, the mixture (60 g) was stirred gently until the ointment formulation was at room temperature. 50 grams was packed in an ointment jar, which was stored in a cool place but not allowed to freeze.

In order to prepare an ointment formulation having active ingredients, the ointment base was prepared as above, except instead of using 29.1 g of pure water, 29.1 g of a plant extract solution (e.g. PRE or tea etc) was used. Solutions of metal salts and Vitamin C were added to the ointment base as required, to prepare the active ointment formulation.

The aqueous formulation was simply a water-based formulation in which the active ingredients (plant extract, metal salt, and in some cases, reducing agent) were dissolved in pure water.

The cream formulation had the following composition: —

Aqueous cream (for 55 grams):

| | |
|---|---|
| Emulsifying component | 16.5 g |
| Phenoxyethanol | 0.55 g |
| Purified water, freshly boiled and cooled | 37.95 g |

Aqueous cream is an emollient and can be used as a base for drugs.

Phenoxyethanol is present as an antimicrobial preservative. It was dissolved in water warmed to 60° C. The emulsifying ointment was weighed and melted on a water bath. Both phases were kept close to 60° C., and then the aqueous phase was added to the melted ointment. The mixture was removed from the heat and stirred continuously until cold. 50 grams was weighed and packed in an ointment jar. The preparation was stored in a cool place but not allowed to freeze.

### Results

FIG. 5 shows a bactericidal assay of an ointment of PRE combined with various components, such as iron or copper salts, and Vitamin C. FIG. 6 shows the same compositions, but their activity after 3 weeks. As shown in FIG. 8, the activity enhancement upon addition of Vitamin C for either Fe(II)/PRE or Cu(II)/Pre compositions is retained for three weeks with no reduction in efficacy for the formulation. Hence, the ointment formulation shown in Table 1, combined with added Vitamin C exhibited greatly enhanced stability compared to the aqueous preparation (data not shown) showing full retention of activity after 3 weeks. Surprisingly, these findings are in sharp contrast to the cream or aqueous formulations, which in the former case had no activity and in the latter lost activity after only 30 minutes. Therefore, the inventor has clearly demonstrated the surprising efficacy, and retained activity over long periods, of ointment-based formulations. This could not have been predicted from previous work.

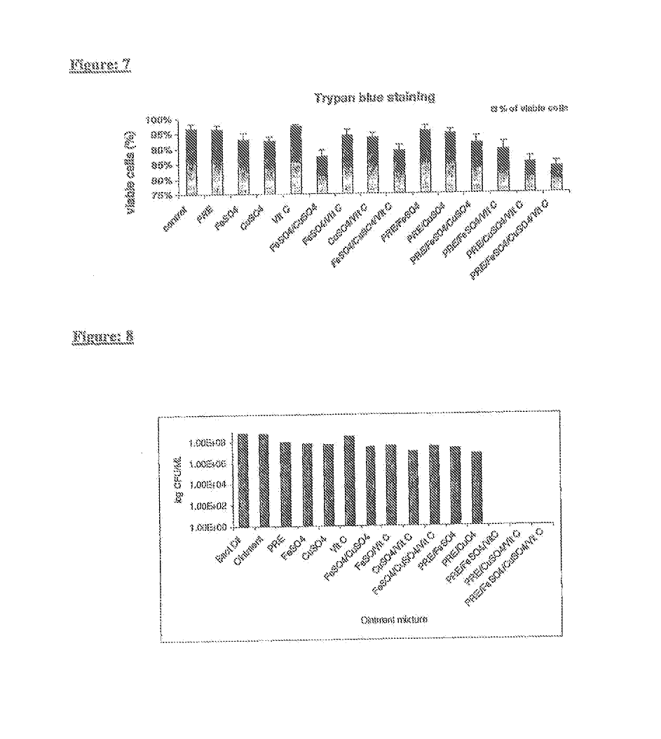

Referring to FIG. 7, there are shown the results of toxicity studies that were carried out using Trypan blue staining. Human breast cancer cells MCF7 were used to examine the effect of pomegranate preparations on mammalian tissues. Trypan blue staining was used to detect non-viable cells (appear blue stained under the microscope). The MCF7 cells were grown to confluence in 75 ml culture flasks using Dulbeccos MEM (Gibco) supplemented with 10% fetal bovine serum (FBS), 25 ug/mL gentamicin and 200 mM L-glutamin (growth medium) and incubated in a 95% air and 5% CO₂ atmosphere at 37° C. Cells were cultured for 5 days prior to treatment with the test substances. Confluent cells were detached with 2 ml of 0.15% trypsin (Sigma) for 5 minutes, 8 ml MEM was added, and the cells were centrifuged at 1000 rpm for 5 min. The supernatant was discarded and the cell pellet was resuspended in 10 ml MEM and counted using a haemocytometer. A 24 well plate was used to seed the cells using a cell concentration of 10⁶/ml (200 ul per well).

Next day the media was removed and the cells were washed with PBS twice, trypsin was added to detach the cells which were then treated with the test substances shown in FIG. 5, incubated for 30 minutes at 37° C. then stained with trypan blue (10 ul of 0.2% was added to 10 ul cell suspension) for 5 minutes, spread onto a microscope slide and covered with a coverslip then examined under the microscope. Non-viable cells appear blue in color because they cannot exclude the dye.

The toxicity studies shown in FIG. 7 revealed that the highest percentage of viable cells was observed with PRE while the lowest was encountered after treating the cells with PRE/FeSO4/CuSO4/Vitamin C combination which is still not very toxic.

### Example 4

Based in the results of Example 3, the inventor focused on enhancing the stability of the product formulation. A wide range of combinations were tested to optimize the efficacy of the active preparation. Final products were identified which retained considerable activities over a five month period. In addition, the inventor carried out further experiments to investigate: —(i) the activation of PRE using copper (II) salts, ii) the activation and enhancement of the PRE/Cu combinations using Vitamin C, and iii) the optimized formulation being an ointment.

### Materials and Methods

The inventor compiled a set of seventeen test preparations for each infectious agent tested. In all, activities were assessed against ten extended spectrum Beta Lactams (ESBL) Pseudomonas aeruginosa. For the most active preparations, a number of formulations were prepared including creams, aqueous preparations and ointments were prepared. These were tested immediately after preparation and in the ensuing months to determine the optimum retention of activity over time.

### Results

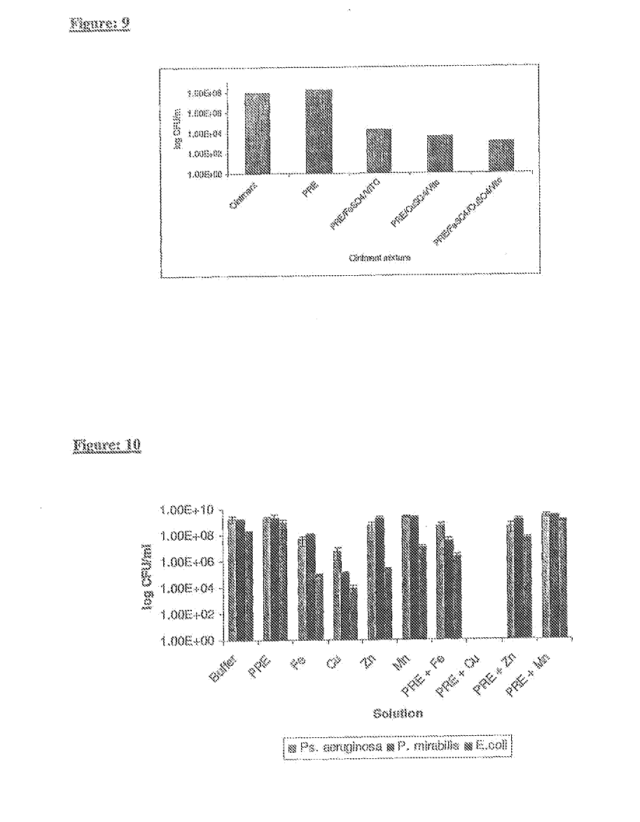

The key objective of this project was to develop a formulation that retained activity to produce an over-the-counter (OTC) preparation for commercial uses. Referring to FIG. 8, there is shown the degree of infectious agent survival after 30 minutes exposure to fresh ointment preparations of test agents shown. Referring to FIG. 9, there is shown infectious agent survival after 30 minutes exposure to ointment preparations of test agents shown after storage at 5° C. for 3 months.

The data show that the ointment reduced cell growth as measures in colony forming units by a factor of 10⁴ compared to the control samples.

As can be seen in FIGS. 8 and 9, the ointment formulation with added Vitamin C exhibited a greatly enhanced activity/stability profile (compared to the aqueous preparation—results not shown) showing full retention of activity after 3 weeks, as discussed in Example 3. In the three month study, as shown in FIG. 9, considerable activities were afforded by the three most active combinations (i.e. PRE/FeSO₄/Vitamin C; PRE/CuSO₄/Vitamin C; and PRE FeSO₄/CuSO₄/Vitamin C). It should be noted that activities without the addition of Vitamin C were less active in the short term and longer term for any preparation and formulation.

### Example 5

The aim of Example 5 was to determine the antimicrobial activities of combinations of pomegranate rind extracts (PRE) with metals salts and Vitamin C against Staphylococcus aureus, Bacillus subtilis, E. coli, Pseudomonas aeruginosa and Proteus mirabilis.

### Materials and Methods

Pomegranate rind extract was prepared by blending 15 grams of PR with 45 mLs distilled water for 10 min. The crude extract was filtered through muslin followed by Whatman No. 1 filtration paper and autoclaved (121° C. for 15 mins) prior to storage at −20° C.

Overnight cultures of the Gram-positive strains (S. aureus, B. subtilis) and the Gram-negative strains (E. coli, Ps. aeruginosa and P. mirabilis) were suspended in Ringer's solution (Oxoid, U.K.) to a turbidity equivalent to 0.5 McFarland (1.5×10⁸ CFU/ml) and 100 μL was spread onto Mueller-Hinton agar plates (OXOID limited, U.K.). The extract (10 μL) was then spotted onto Whatman no 1 filter paper (5 mm diameter). Plates were incubated at 37° C. for 24 h, after this time the diameter of the zone of inhibition was recorded.

All reagents were purchased from Sigma-Aldrich (Poole, Dorset) and distilled water was used throughout. Overnight cultures on nutrient agar were then suspended in Ringer's solution (Oxoid, U.K.) to a turbidity equivalent to 0.5 McFarland (1.5×10⁸ CFU/ml). An aliquot of the PRE extract (330 μl) was added to 700 μl of the freshly prepared solutions (4.8 mM) of metal salts (FeSO₄, CuSO₄, MnSO₄, ZnO). The final solution was protected from light (Stewart et al. 1998. J. Appl Microbiol 84:777-783).

The appropriate bacterial dilution was prepared and 50 μl placed in a sterile Eppendorf micro-centrifuge tube with a 100 μl of the extract/metal salt solution. After exposure of the bacteria for 30 minutes at room temperature, the activity of the bactericidal agent was neutralized by adding an equal volume of 2% (v/v) Tween-80 (Sigma Chemical Co., UK) in Lambda buffer. Serial dilutions were prepared in Ringer's solution (10⁻⁵), 10 μl of each dilution is spotted on nutrient agar plate and incubated for 24 hours at 37° C. Each assay was conducted in triplicate.

The assay was carried out as described above with the following addition: Vitamin C was added to the metal ion (FeSO₄, CuSO₄) solution immediately prior to mixing with the PRE. Aliquots of Vitamin C were made to give final metal ion:Vitamin C ratios (and Vitamin C concentrations) of 1:1 (4.8 mM), 1:5 (24 nM), 1:20 (96 mM) (metal salt:Vitamin C). 700 μl of this solution was then added to PRE.

### Fractionated PRE Assay

PRE extracts were fractionated by molecular weight using Millipore ultra-filtration devices (nominal M. Wgt. cut-off=5,000 a.m.u.) and the resulting extracts were tested by the disc diffusion method outlined above.

### Results

Using the disk diffusion method antimicrobial efficacies were examined against a panel of five microbes. Maximum activities for PRE were observed against the S. aureus and B. subtilis. Moderate effects (zone sizes) were seen against Ps. aeruginosa and P. mirabilis and there was little activity against E. coli. The average zone diameters were 17 mm, 14 mm, 9 mm and 8 mm for S. aureus, B. subtilis, Ps. aeruginosa and P. mirabilis respectively.

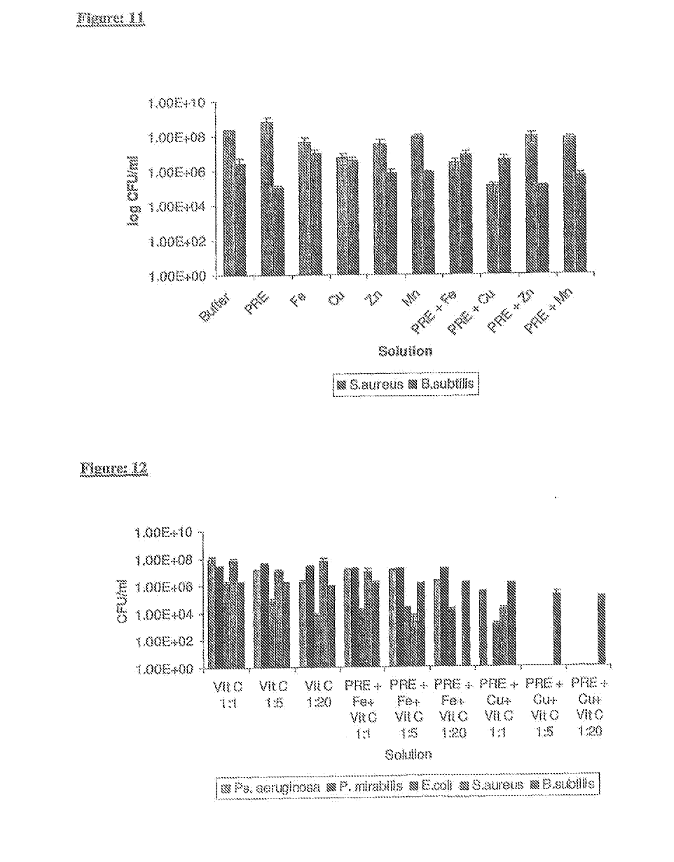

The antimicrobial activities of PRE extracts along with the metal salt additives were assessed using a modified version of the adopted by Stewart et al. (1998) for observing the additive effects of metal ions. PRE alone did not exhibit antimicrobial activity against the majority of the bacteria tested; except B. subtilis, which may be due to the short incubation time of 30 minutes. Against the Gram-negative isolates the metal ions showed moderate activity, the greatest results were seen against E. coli (as shown in FIG. 10) with Cu (II) ions reducing the cell survive population by a factor of 10⁴ compared to the buffer. The most striking result was seen with the combination of the PRE with cupric salts where no detectable growth was seen. Moderate antimicrobial activity was seen with Cu (II) ions and PRE in combination against S. aureus that reduced the surviving population by circa 10³ compared to the buffer (as shown in FIG. 11).

Further studies were conducted to enhance the activity of the PRE/metal ion combination and to elucidate the mechanism of action. Based upon a putative oxidative damage mechanism afforded by the redox active metal ion, the inventors assessed the antimicrobial efficacy upon addition of the reductant Vitamin C. Owing to the high activity exhibited for the PRE/Cu(II) combination against all Gram-negative isolates, the inventors studied the addition of Vitamin C to the PRE/Fe(II) combinations (as shown in FIG. 12). For E. coli, a decrease in growth of 10² was seen with the addition of a stoichiometric equivalent of Vitamin C (with respect to metal ion concentration). Addition of 5 and 20 equivalents of Vitamin C resulted in a reduction in growth of 10³ and no discernable growth respectively. For S. aureus, addition of Vitamin C to the PRE/Cu(II) mixture had no significant effect at one equivalent but a marked effect at 5 and 20 equivalents of Vitamin C (no detectable growth in either).

In order to investigate the mode of antimicrobial action, the PRE was subjected to fractionation on the basis of nominal molecular weights. The fraction with a nominal MW below 5,000 was compared to the untreated PRE assessed using the disc diffusion method. As shown in Table 2, similar activities were exhibited by the low molecular weight fraction in comparison to the whole PRE.

TABLE 2
Diameter of the zones of inhibition of the low
molecular weight fraction of PRE compared to whole
PRE (±SEM) against a panel of five bacteria.

| | Low Molecular | |
|---|---|---|
| | Weight Fraction <5000 | |
| Organism | Da | Whole PRE fraction |
| S. aureus | 11 ± 0.25 | 14 ± 0.14 |
| B. subtilis | 13 ± 0.09 | 15 ± 0.14 |
| Ps. Aeruginosa | 8 ± 0.09 | 8 ± 0.14 |
| P. mirabilis | 7 ± 0.00 | 7 ± 0.00 |
| E. coli | 0 | 0 |

In this study, metal salts were applied to further enhance the properties of pomegranate. Preliminary results using the disk diffusion assay, to assess antimicrobial activity against a panel of bacteria, showed that the PRE was most active against the Gram-positive organisms (S. aureus and B. subtilis). Disc diffusion assessment of the low molecular weight fraction of PRE suggest the antimicrobial component(s) of PRE are found within <5000 Da portion of the extract. However, in the suspension assay, the PRE alone showed little or no antimicrobial activity against any of the bacteria tested, perhaps due to the short incubation time.

For the Gram-negative bacteria the combination of PRE:Cu(II) gave the best results with no detectable growth observed with all three isolates after 30 mins. For S. aureus the addition of 5 and 20 equivalents of Vitamin C to the PRE:Cu(II) result in no detectable growth after 30 mins. The addition of Vitamin C to PRE:Fe(II) also resulted in a reduction in growth for S. aureus of circa 10⁴log₁₀.

In conclusion, PRE in combination with Cu(II) ions exhibit dramatic synergistic antimicrobial effects against E. coli, Pseudomonas aeruginosa and Proteus mirabilis and moderate activity against S. aureus. The active component(s) in the PRE are found in the low molecular weight fraction. The addition of high quantities of Vitamin C markedly enhanced the activities of both PRE/Fe(II) and PRE/Cu(II) mixtures against at least S. aureus.

### Example 6

The aim of this Example was to explore the potential role for metal ions in enhancing the activities of PRE against clinical isolates of S aureus. Thirty isolates were tested which include 10 MRSA (methicillin resistant S. aureus), 10 MSSA (multiple antibiotic-resistant methicillin resistant Staphylococcus aureus) and 10 Panton-Valentine Leukocidin (PVL) producing cMRSA isolates (community acquired MRSA, which produce Panton-Valentine leukocidin). The example demonstrates the antimicrobial activities of pomegranate rind extracts (PRE) against Staphylococcus aureus (MSSA), MRSA and PVL positive cMRSA. For MRSA and MSSA strains, exposure to copper (II) ions for 2 hours had moderate activities of between 10² to 10³ log₁₀ reduction in growth, which was enhanced by the addition of PRE to 10⁴ log₁₀ reduction in growth observed in 80% of the isolates. However, the PVL positive cMRSA strains were surprisingly more sensitive to copper (II) ions and had moderate activities of between 10³ log₁₀ reduction in growth for 60% of the isolates.

### Materials and Methods

Pomegranate rind extract (PRE) was prepared firstly by cutting rind into small squares (approximately 5 mm²) which were dried at 55° C. for 24 hours, and stored in an air tight container in the dark until further use. 10 g of dry rind was added to 150 ml distilled water and place in a shaker (at 80 rpm) at room temperature for 24 hours. The crude extract was passed thought muslin and a Whatman filter No. 1 to remove the particulate matter, prior to filter sterilizing by passing through a 0.2 um filter (Millipore), into a sterile bottle. The extract was stored at −20° C. for future use.

Clinical isolates of methicillin resistant (n=10), methicillin sensitive (n=10) Staphylococcus aureus (MRSA and MSSA) and Panton-Valentine Leukocidin producing cMRSA (n=10) were used in the study. The MRSA and MSSA isolates were collected from the Royal Marsden Hospital (London, UK) and the cMRSA isolates were collected from the Devon and Exeter Hospital (UK). The isolates were cultured over night on nutrient agar (Oxoid), aerobically at 37° C. and then frozen in cyrovials (Pro-labs) at −80° C. until required. Prior to use all isolates were passaged twice on nutrient agar aerobically at 37° C. In all assays culture were prepared by using overnight cultures on nutrient agar that were then suspended in Ringer's solution (Pro-Lab, U.K.) to a turbidity equivalent to 0.5 McFarland (1.5×10⁸ cfu ml⁻¹).

All reagents were purchased from Sigma-Aldrich (Poole, Dorset) and distilled water was used as a chemical diluent throughout. The method used was an adaptation of that described by Stewart et al (1998) supra. Briefly, overnight cultures on nutrient agar were then suspended in Ringer's solution (Oxoid, U.K.) to a turbidity equivalent to 0.5 McFarland (1.5×10⁸ CFU/ml). An aliquot of the PRE extract (330 μl) was added to 700 μl of the freshly prepared solutions (4.8 mM) of metal salts (FeSO₄, CuSO₄); the final solution was protected from light (Stewart et al. 1998).

The appropriate bacterial dilution was prepared and 50 μL it placed in a sterile Eppendorf micro-centrifuge tube with a 100 μL of the extract/metal salt solution. Following treatment of the bacteria for 2 hours at room temperature, the activity of the bactericidal agent was neutralized by adding an equal volume of 2% (v/v) Tween-80 (Sigma Chemical Co., UK) in Lambda buffer. Serial dilutions were prepared in Ringer's solution (10⁻⁵), 10 μL of each dilution is spotted onto nutrient agar plate and incubated aerobically for 24 hours at 37° C. Each assay was carried out in triplicate.

The antimicrobial assay was carried out as previously stated with the following modification. Before adding the metal salts solution to PRE, Vitamin C was added to the metal salts. Varying concentrations of Vitamin C were added comprising the following ratios; 1:1 (4.8 mM), 1:5 (24 nM), 1:20 (96 mM) (metal salt:Vitamin C) was added to the metal solution, 700 μL it of this solution was then added to PRE.

Micro-dilution plates were prepared with freeze dried PRE or CuSO₄ which was added to sterile water in a concentration of 800 mg/ml. The plates were prepared as follows, 50 μl of four-times strength Iso-Sensitest broth was added to the first row of wells and 50 μl of double strength Iso-Sensitest broth was added to all remaining wells. To the first row of wells 50 μl of the PRE was added and mixed, 50 μl of broth from row A was transferred to row B and mixed, this process was continued to row E Finally, 50 μl of broth was removed from well F and discarded. Then the overnight cultures were suspended in Ringers solution to a turbidly of 0.5 McFarland (1.5×10⁸ cfu/ml). 50 μl of suspension were added to well A (final concentration of PRE in well A=200 mg/ml) through to G All samples were carried out in Triplicate. All plates were incubated at 37° C. for 24 hours. After incubation 10 μl of broth from each well was spotted onto nutrient agar and incubated at 37° C. for 24 hours. After incubation the plates were examined to determine breakpoints by the presence or absence of growth.

The assay was carried out as above with the following changes: PRE and CuSO₄ were prepared as before but using four times concentration of half the determined MIC (ie. If the MIC was 4 mg/ml, half this would be 2 mg/ml and therefore stock concentration would be 8 mg/ml). Addition the CuSO₄ was made to the PRE suspension instead of sterile water.

### Results

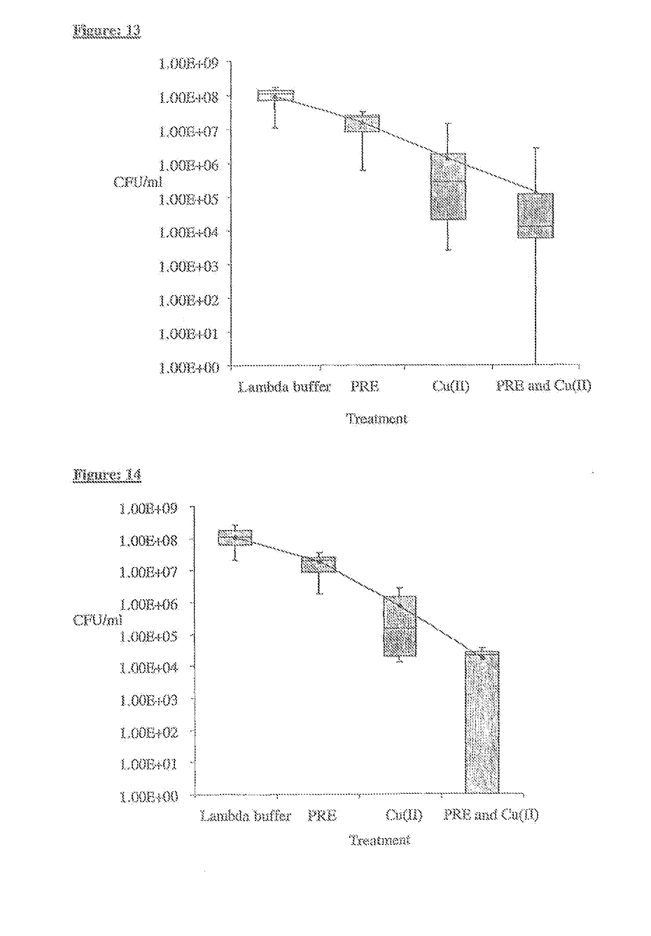

The suspension test method outlined by Stewart et al (1998) J. Appl Microbiol 84:777-783, was adapted to assess the antimicrobial activities of PRE extracts along with the cupric salts. For the MRSA isolates, the PRE on its own, had marginal activity against all isolates studied (as shown in FIG. 13). In contrast, the copper (II) ions had moderate activities of between 10² to 10³ log reduction in growth. However, in combination the PRE/copper(II) mixture surprisingly exhibited an enhanced activity of 10⁴ log₁₀ reduction in growth which were observed in 80% of the isolates. Similar results were observed for the MSSA isolates, with no real effect for the PRE alone, but addition of Cu(II) ions affording enhancement of antimicrobial activity for 80% of isolates by 10⁴ log orders (as shown in FIG. 14).

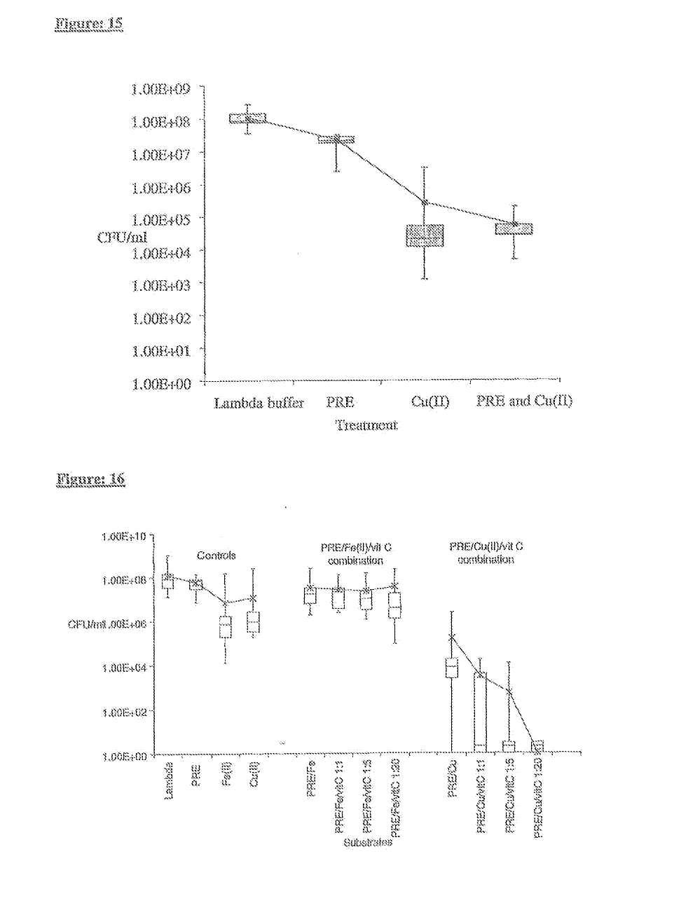

For the PVL positive cMRSA isolates, the PRE on its own, had marginal activity against all isolates studied. In contrast to the MSSA and MRSA, the PVL positive cMRSA isolates were even more sensitive to copper (II) ions and had moderate activities of between 10³ log reduction in growth for 60% of the isolates. Notably, for 40% of the isolates less reduction in growth indicated less sensitivity to Cu(II) ions, however, addition of PRE reduced the growth in these 40% in line with the copper-sensitive 60% (as shown in FIG. 15).

Determination of the MIC of PRE and CuSO₄ individually and in combination are shown in Table 3.

TABLE 3
Minimum inhibition concentration of PRE and CuSO₄ alone
and in combination against ten isolates each of MRSA,
MSSA and Panton-Valentine Leukocidin producing cMRSA.

| Isolates | No. of | MIC (mg/ml) | | |
|---|---|---|---|---|
| type | isolates | PRE | CuSO4 | PRE/cuSO4 |
| MRSA | 1 | 12.5 | 3.125 | 6.25/1.563 |
| | 1 | 25 | 1.563 | 6.25/0.781 |
| | 2 | 25 | 3.125 | 12.5/1.563 |
| | 3 | 25 | 1.563 | 12.5/0.781 |
| | 2 | 12.5 | 1.563 | 6.25/0.781 |
| | 1 | 12.5 | 1.563 | 6.25/0.781 |
| MSSA | 1 | 25 | 1.563 | 6.25/0.391 |
| | 3 | 25 | 1.563 | 12.5/0.781 |
| | 1 | 25 | 3.125 | 6.25/0.781 |
| | 1 | 12.5 | 1.563 | 3.125/0.391 |
| | 1 | 12.5 | 0.782 | 3.125/0.196 |
| | 3 | 12.5 | 1.563 | 6.25/0.781 |
| PVL | 3 | 25 | 1.563 | 6.25/0.391 |
| | 4 | 25 | 3.125 | 6.25/0.781 |
| | 2 | 25 | 0.781 | 6.25/0.195 |
| | 1 | 25 | 1.563 | 12.5/0.781 |

PRE had an MIC between 25-12.5 mg/ml for all isolates tested. The combination of PRE:Cu(II) against all isolates of S. aureus resulted in values which were half or a quarter of the MIC of PRE or CuSO₄ alone. Thus, a considerable additive effect is seen against S. aureus for the combination.

In conclusion, PRE in combination with Cu(II) ions exhibit surprisingly synergistic antimicrobial effects against three classes of S. aureus. For MSSA, MRSA and PVL positive cMRSA isolates, antimicrobial activities were exhibited by the mixture. The inventors believe that they are the first to report of the efficacy of pomegranate against PVL positive cMRSA isolates.

### Example 7

Example 7 demonstrates the antimicrobial activities of pomegranate rind extracts (PRE) in combination with Fe(II) and Cu(II) salts against multi-drug resistant (eg extended spectrum β-lactamase) Pseudomonas aeruginosa. Marked activities were observed for the aqueous PRE:Cu preparations which were greatly enhanced by addition of the reductant Vitamin C. An ointment preparation of the PRE:Fe(II):Vitamin C system showed moderate activity which was exceeded by the corresponding Cu(II) preparation over a three months period.

### Materials and Methods

Pomegranate rind extracts (PRE) were prepared by cutting the rind into small cubes (approximately 5 mm³) which were dried at 55° C. for 24 hours. Dried rind was stored in air tight containers in the dark until further use. Stock solutions were prepared by adding 10 g of dry rind to 150 ml distilled water and shaking (at 80 rpm) at room temperature for 24 hours. The crude extract was passed through muslin and a Whatman filter No. 1 to remove the particle matter, and filter sterilised by passing through a 0.2 um filter (Millipore) into a sterile bottle. The PRE stock solutions were stored at −20° C.

Clinical isolates of multi-drug resistant Pseudomona aeruginosa (ESβL P. aeruginosa), were collected at the Royal Marsden Hospital (Sutton, UK). The isolates were grown overnight on nutrient agar (Oxoid, UK) and then frozen in cyrovials (Pro-labs, UK) for future use.

All reagents were purchased from Sigma-Aldrich (Poole, Dorset) and distilled water was used throughout. P. aeruginosa isolates were removed from the freezer and first passaged on nutrient agar twice before use. Overnight cultures on nutrient agar were then suspended in Ringer's solution (Oxoid, U.K.) to a turbidity equivalent to 0.5 McFarland (1.5×10⁸ CFU/ml). An aliquot of the PRE extract (330 ml) was added to 700 ml of the freshly prepared solutions (4.8 mM) of metal salts (FeSO₄, CuSO₄,). The final solution was protected from light (Stewart et al. 1998).

The appropriate bacterial dilution was prepared and 50 μl placed in a sterile Eppendorf micro-centrifuge tube with a 100 μl of the extract/metal salt solution. After exposure of the bacteria for 30 minutes at room temperature, the activity of the bactericidal agent was neutralized by adding an equal volume of 2% (v/v) Tween-80 (Sigma Chemical Co., UK) in Lambda buffer (Stewart et al. 1998). Serial dilutions were prepared in Ringer's solution (10⁻⁵), 10 μl of each dilution is spotted on nutrient agar plate and incubated for 24 hours at 37° C. Each assay was conducted in triplicate.

The assay was carried out as described above with the following addition: Vitamin C was added to the metal ion solution immediately prior to mixing with the PRE. Aliquots of Vitamin C were made to give final metal ion:Vitamin C ratios (and Vitamin C concentrations) of 1:1 (4.8 mM), 1:5 (24 nM), 1:20 (96 mM) (metal salt:Vitamin C). 700 μl of this solution was then added to PRE.

Two formulations were prepared and tested: a hydrous ointment and an aqueous cream. The composition of the hydrous ointment base was prepared as followed (for 60 grams) 30 g wool alcohol ointment, 0.6 g phenoxyethanol, 0.3 dried magnesium sulphate, 29.1 g purified water. The components were mixed until they formed a smooth ointment. The composition of the aqueous cream base was 150 g emulsifying ointment, 5 g phenoxyethanol and 345 g purified water. The components were mixed until they formed a smooth cream. For both formulations containing PRE, the base preparation was carried out as above. However, PRE was used instead of water, and solutions of metal salts and Vitamin C were added to the base formulation.

The assay was carried out as above with the following changes. 0.5 g of either ointment or cream was first added to 10 ml of sterile water and vortex until dissolved in water. The appropriate bacterial dilution (1.5*10⁸CFU/ml) was prepared and 50 μl was placed in a sterile Eppendorf micro-centrifuge tube with a 100 μl of the formulation solution, which was assayed as described above. Formulations were stored in the dark at 5° C. and for 3 months, prior to re-testing to determine loss in activity.

### Results

Nine multi-drug resistant clinical isolates of Pseudomonas aeruginosa were subjected to challenge by PRE alone or in combination with metal ions. FIG. 16 gives the results of the suspension test method used to assess the antimicrobial activities of PRE extracts along with cupric and ferrous salts. For control samples, PRE alone had no significant effect and both Fe(II) and Cu(II) treatments resulted in a modest ca 10¹ log¹⁰ reduction in growth (mean values). A minor reduction in growth (ca 10¹ log₁₀) was observed upon treatment with the PRE:Fe(II) combination. As expected, treatment with Cu(II) alone resulted in a reduction in growth of ca 10² log₁₀. However, in contrast to the result with PRE:Fe(II) where marginal effect was seen, Vitamin C addition greatly enhanced the activities of the PRE:Cu(II) combinations. Addition of one equivalent of Vitamin C enhanced the growth retardation of PRE:Cu(II) from ca 10⁵ to ca 10³ log₁₀ reductions. Five equivalents of Vitamin C afforded a 10⁴ log₁₀ reduction in growth and for 20 equivalents of Vitamin C no detectable growth was observed.

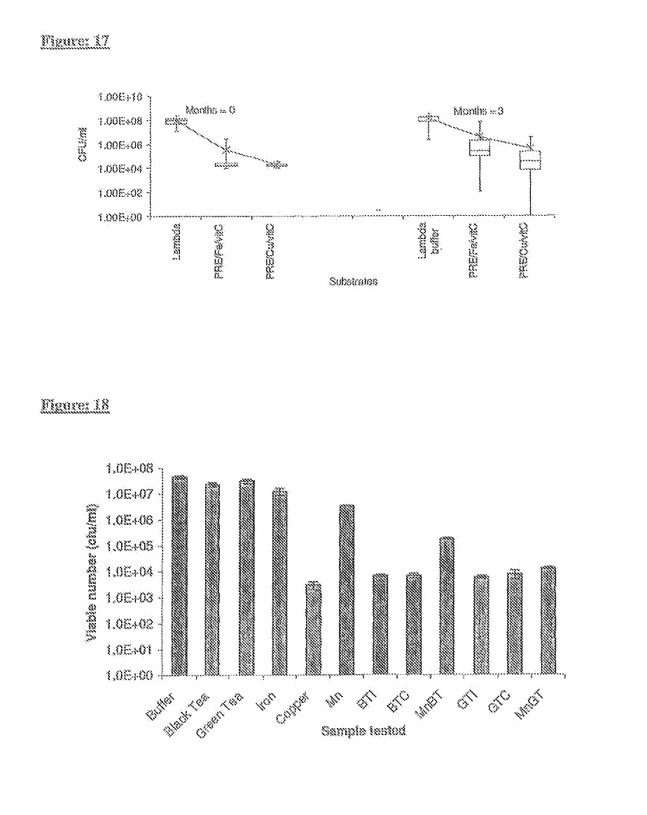

The results of the suspension test method used to assess the antimicrobial activities of ointment formulations are given in FIG. 17. The ointment PRE:Fe(II) combination with added Vitamin C gave a reduction in growth of 10² log₁₀ in contrast to the corresponding aqueous formulation which exhibited no significant activity (as shown in FIG. 16). In the case of the Cu(II):PRE combination with Vitamin C, the expected 10⁴ log₁₀ reduction in growth was observed in line with the results for the aqueous formulation (as shown in FIG. 16). After storage for three months, it is notable that a similar pattern of activity is retained for each combination with a slight loss of activity of ca 10¹ log₁₀ reduction in growth for both combinations. The cream based formulations were found to be considerably less active and less stable.

As shown in FIG. 16, and in line with the results obtained by Stewart et al. (1998) supra, the PRE:Fe(II) system had negligible effect on the bacterial growth with a modest retardation of growth occurring for Fe(II) treatment alone. The lack of antimicrobial activity exhibited by the PRE:Fe(II) combination may arise from the instability of ferrous ions in aerated aqueous solutions. However, in contrast, the PRE:Cu(II) combination exhibited a ca 10² log₁₀ reduction compared to Cu(II) treatment alone.

Following this result, a further investigation involved addition of the reductant Vitamin C to explore if the mechanism was attributable to redox-cycling. The profound effects produced upon addition of Vitamin C indicated that reducing the metal ion may be important. It is notable that in the aqueous preparations this enhanced effect was mainly seen for the PRE:Cu(II) combination. A complete retardation in growth was observed for the PRE:Cu(II) system on addition of 20 equivalents of Vitamin C. These results suggest that the combination of PRE:Cu and Vitamin C may be a possible antimicrobial agent for treating multi-drug resistant (eg ESβL) Pseudomonads that are becoming an increasingly resistant organism to currently available antibiotics.

The key requirement for the development of a stable formulation arose as the aqueous combinations were found to have a short active shelf life. Initial stability and activity tests were conducted on aqueous, cream and ointment formulations with the focus moving to the ointment as it had the optimal properties. In addition to developing a stable and active ointment formulation of the PRE:Cu(II) Vitamin C, the inactivity of the aqueous preparation of the PRE:Fe(II):Vitamin C system is reversed and stable as an ointment.

In conclusion, the combination of aqueous solutions of PRE and Cu(II) salts show antimicrobial activity against Pseudomonads which is further enhanced with addition of Vitamin C. Stable and active ointment formulations have been developed for combinations of both PRE:Cu(II) and PRE:Fe(II) systems with Vitamin C. The inventors believe that they are the first to report of the activation of a natural product by addition of a redox-active metal ion along with the reductant Vitamin C.

### Example 8

The aim of Example 8 was to establish the optimum extraction method for green and black tea and determine the efficacy of these extracts against Staphylococcus aureus, Pseudomonas aeruginosa and Proteus mirabilis in the presence and absence of metal ions.

### MATERIALS AND METHODS

Isolates of Staph. aureus, Prot. mirabilis and Ps. aeruginosa were maintained on Brain Heart Infusion slopes (Oxoid Ltd), at room temperature, until used. Prior to use, the organisms were inoculated into 5 mL aliquots of Brain-heart infusion broth (Oxoid Ltd) and incubated aerobically overnight at 37° C. These starter cultures were used as inocula for the screening assays.

A preliminary screen (disc diffusion method) against Staph. aureus was established to investigate the optimum method of extraction. Tea extracts are prepared from Sencha green Chinese tea and Yorkshire black tea obtained from a commercial outlet. The methods employed were hot and cold water extraction: 8 g of loose leaf green or black tea were infused with 100 mL of sterilized distilled water at 100° C. or ambient temperature for recorded time intervals up to one hour in the dark. Overnight bacterial cultures were suspended in Ringer's solution to a cell concentration of 1×10⁵ CFU/mL and swabbed evenly in three directions on Mueller-Hinton agar plates (Oxoid Limited). The plates were then spotted with 10 μL of each test extract prior to aerobic incubation at 37° C. for 24 h.

The method outlined above was used to examine the effects of pH and extract concentration on antimicrobial activity. The optimum tea pH was determined using 8 g of tea leaves per 100 mL water at 100° C. for 10 min. The pH values of untreated tea extracts were in the range of 4.5 to 5.0 pH units. These were adjusted using aqueous solution of NaOH (1.0 molar) by careful drop wise addition to reach the desired pH values (+/−0.3) of 5, 6, 7, 8, 9. The concentration effects for both green and black leaves were examined using the hot water extraction method (infusion between 10 and 20 minutes) for 4, 6, 8 and 10 g in 100 mL.

Tea infusions were prepared as previously with boiling sterilized distilled water. After 10 minutes, the extracts are removed from the beaker into sterilized universal tubes. The pH of these extracts was neutralized using a 1 M NaOH solution. The extracts are refrigerated, used within 4 days and without further treatment.

Overnight cultures of the Gram-positive strain (Staph. aureus) and the Gram-negative strains (Ps. aeruginosa and Prot. mirabilis) were suspended in Ringers solution to a turbidity equivalent to 0.5 McFarland (1.5×10⁸ CFU/ml). Metal salts were added to the optimum tea extracts immediately before the assay was conducted. The tea extract (330 μl) was added to 700 μl of the freshly prepared ferrous sulphate or cupric sulphate solution (4.8 mmol metal salt; pH 6.3); the final solution was protected from light.

The appropriate bacterial dilution was prepared and 50 μl of that was placed in a sterile Eppendorf micro-centrifuge tube with a 100 μl of the extract/metal salt solution. Following 30 min incubation at room temperature, the activity of the putative bactericidal agent was neutralized by adding an equal volume of 2% (v/v) Tween-80 (Sigma Chemical Co., UK) in Lambda buffer (Stewart et al., 1998). Serial dilutions were prepared in Ringer's solution, 10 μL aliquots of each dilution were spotted onto nutrient agar plates and incubated aerobically for 24 hours at 37° C. Each assay was conducted in triplicate.

### Results

Studies on the optimum extraction methodology for the black and green teas established the best method was extraction at 100° C. for 10 minutes. Preliminary investigations ascertained the mean zones of inhibition (in mm for triplicate runs) for green tea varied with pH, showing optimal activities at pH 7/8 with inhibition values of 16, 18, 21, 21, 17 mm for the pH values of 5, 6, 7, 8, 9 respectively. Similarly, black tea exhibited optimal activity at pH 5/7 with values of 25, 27, 26, 17, 16 mm at pH 5, 6, 7, 8, 9 respectively. In both cases, a negative trend in activity is evident in basic conditions. The investigations of the effects of pH showed the value of pH7 to be optimal against Staph. aureus.

The antimicrobial activities of black and green tea extracts along with the metal salts additives against Staph. aureus are shown in FIG. 18. The data show poor efficacy for both types of tea extract and for the iron and manganese salts when tested independently. Upon addition of the cupric salt a bactericidal efficacy (equating to a reduction in CFU/mL by 10⁴) was observed. For both black and green tea extracts the addition of cupric ions slightly diminished the effect compared to the cupric salt alone. On the addition of manganese to black tea, a minimal reduction in viability was established (reduction of 100 CFU/mL) which was doubled in the presence of green tea. In contrast, there was an enhancement of inhibitory activity against Staph. aureus with the addition of ferrous ions to both tea extracts, with a reduction in CFU/ml of circa 10⁴ for each, equivalent to the level seen on action of cupric ions alone.

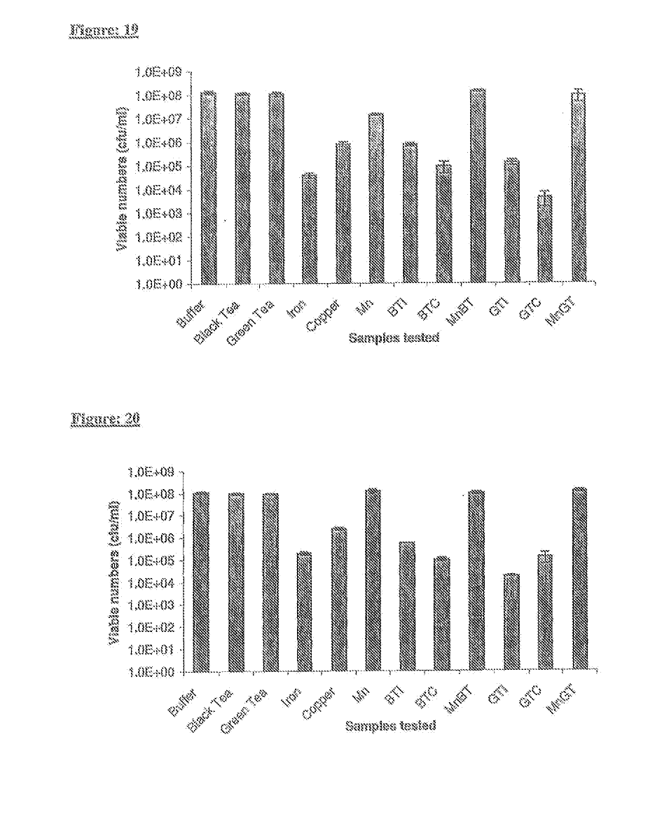

Similar antimicrobial profiles were determined for tea extracts along with the metal salts additives against Prot. mirabilis and Ps. aeruginosa as shown in FIGS. 19 and 20. The data shows poor efficacy for both types of tea extract when tested independently as seen with Staph. aureus. For the ferric and cupric salts, moderate bactericidal efficacies (equating to a reduction in CFU/mL by 10³ and 10² respectively) was observed, whilst the reduction in the presence of manganese was a factor below that seen with cupric salts. Upon addition of ferric ions to black tea extracts a decrease in efficacy was observed on comparison to the ferric salt solution alone with Prot. mirabilis, whereas there was no noteworthy change in efficacy for Ps. aeruginosa. In the case of the green tea extract when combined with the ferric salt, an equivalent efficacy to the ferric salt alone was observed. The action of manganese salts on their own with both Gram-negative microorganisms was found to be minimal. In the presence of both green and black tea no response was elicited from the inclusion of manganese ions against Pseudomonas aeruginosa. However, a slight antagonistic effect was observed against Prot. mirabilis in the same circumstances.

These data demonstrate that significant enhancement of natural product extracts as antimicrobial agents can be achieved by addition of redox active metal ions. It is notable that contrasting activity profiles are seen for different microbial strains with green tea extract combined with cupric/ferrous/manganese salts exhibiting least variation with the Gram-positive. This example highlights the significance of natural extracts and their enhanced efficacy against microorganisms of medical importance in the presence of redox active metal ions at a time when alternative strategies for the control of microorganisms causing hospital acquired infection (HAI's) are being sought.

The inventor has demonstrated in the Examples that the activity of iron-based PRE compositions may be augmented and prolonged upon combination with a reducing agent, such as Vitamin C. This was surprising given that neither the mechanism of action nor the reason for the very short activity lifespan was understood for iron-based compositions before the inventor began his research. Furthermore, the inventor has shown that, surprisingly, other metal ion-based (eg copper) PRE compositions also exhibit considerable antimicrobial activity. The mechanism of action of copper-based compositions is not fully understood. However, it is believed that the mechanism for copper-based compositions is different to that of iron-based compositions. A significant advantage of using copper as opposed to iron is that the composition does not turn black. It will be appreciated that a composition which turns black would be undesirable for topical use.

In addition to the isolation and identification of new formulations with long term activities, applications against hospital acquired infections have been demonstrated opening up further markets for exploitation of the compositions in accordance with the invention. The identification of new additives (Copper(II) ions and Vitamin C) along with exemplification of the optimum formulation for long term efficacy present considerable commercial advantages over the known compositions disclosed in EP 0,744,896B1.

While the invention has been described in connection with certain embodiments, it is to be understood that the invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law.

1. An antimicrobial composition comprising:
(i) a copper salt and/or a cobalt salt and/or a nickel salt; and
(ii) an extract of a plant selected from a group consisting of Punica granatum, Viburnum plicatum, Camellia sinensis, and Acer spp.

2. (canceled)

3. A composition according to claim 1, wherein the composition comprises a reducing agent.

4. An antimicrobial composition comprising:
(i) a copper salt and/or an iron salt and/or a nickel salt and/or a cobalt salt;
(ii) an extract of a plant selected from a group consisting of Punica granatum, Viburnum plicalum, Camellia sinensis, and Acer spp.; and
(iii) a reducing agent.

5.-6. (canceled)

7. A composition according to claim 2, wherein the reducing agent comprises cysteine, glutathione or Vitamin C.

8.-10. (canceled)

11. A composition according to claim 1, wherein the concentration of the copper, nickel, or cobalt salt is in the range of about 0.1 mM to about 200 mM.

12.-13. (canceled)

14. A composition according to claim 1, wherein the composition comprises a copper (II) salt.

15.-21. (canceled)

22. A composition according to claim 1, wherein the composition comprises an extract of Punica granatum.

23. A composition according to claim 1, wherein the composition comprises pomegranate rind extract.

24. (canceled)

25. A composition according to claim 1, wherein the composition is provided as an ointment formulation.

26. A solid or liquid concentrate, which on dilution with water, provides a composition according to claim 1.

27.-45. (canceled)

46. A method of treating, preventing or ameliorating a microbial infection, the method comprising administering to a subject in need of such treatment a therapeutically effective amount of a composition according to claim 1.

47. A composition according to claim 3, wherein the reducing agent comprises cysteine, glutathione or Vitamin C.

48. A composition according to claim 3, wherein the concentration of the copper, nickel, or cobalt salt is in the name of about 0.1 mM to about 200 mM.

49. A composition according to claim 3, wherein the composition comprises a copper (II) salt.

50. A composition according to claim 3, wherein the composition comprises an iron (II) salt.

51. A composition according to claim 3, wherein the composition comprises an extract of Punica granatum.

52. A composition according to claim 3. Wherein the composition comprises pomegranate rind extract.

53. A composition according to claim 3, wherein the composition is provided as an ointment formulation.

54. A solid or liquid concentrate, which on dilution with water, provides a composition according to claim 3.

55. A method of treating, preventing or ameliorating a microbial infection, the method comprising administering to a subject in need of such treatment a therapeutically effective amount of a composition accordion to claim 3.
